(19) United States
(12) Patent Application Publication
(10) Pub. No.: US 2021/0047652 A1
(43) Pub. Date: Feb. 18, 2021

(54) METHODS FOR GENETIC TRANSFORMATION AND GENOME MODIFICATION IN LEGUMES

(71) Applicant: Regents of the University of Minnesota, Minneapolis, MN (US)

(72) Inventors: Feng Zhang, Maple Grove, MN (US); Robert M. Stupar, Arden Hills, MN (US); Daniel F. Voytas, Falcon Heights, MN (US); Gunvant Patil, St. Paul, MN (US)

(21) Appl. No.: 16/913,478

(22) Filed: Jun. 26, 2020

Related U.S. Application Data
(60) Provisional application No. 62/867,173, filed on Jun. 26, 2019.
(60) Provisional application No. 62/877,456, filed on Jul. 23, 2019.

(51) Int. Cl. C12N 15/82; C12N 15/66

(52) U.S. Cl. CPC C12N 15/8241; C12N 15/8201; C12N 15/66; C12N 15/8216

(57) ABSTRACT

Methods and materials that can be used to achieve genetic transformation of legumes are provided herein. For example, materials and methods for transforming whole plants using a hairy root-like system that is not species or genotype-dependent are described.

### CROSS-REFERENCE TO RELATED APPLICATIONS

This application claims benefit of priority from U.S. Provisional Application No. 62/877,456, filed Jul. 23, 2019, and U.S. Provisional Application No. 62/867,173, filed Jun. 26, 2019. The disclosures of the prior applications are considered part of (and are incorporated by reference in) the disclosure of this application.

### TECHNICAL FIELD

This document relates to methods and materials that can be used to achieve genetic transformation of legumes.

### BACKGROUND

Legumes are a large, diverse family of nitrogen fixing plants. Crop legumes are grown agriculturally, primarily for human consumption, livestock forage, and soil-enhancing green manure. Extensive efforts have been made to improve agronomically important traits in crop legumes through both traditional breeding and genetic engineering. However, the lack of efficient plant transformation methods has been a major limitation in applying biotechnology tools towards trait development in crop legume species. Many legume crops, including common bean, pea, chickpea, cowpea, pigeon pea, peanut, ground nuts, and many soybean varieties, are recalcitrant to plant genetic transformation. In the past 40 years, researchers have put significant effort into improving plant tissue culture and transformation processes by optimizing factors such as growth media, exogenous hormone application, explant type, and delivery method, including Agrobacterium-mediated delivery, ballistic gene gun delivery, and nanoparticle delivery (Altpeter et al., Plant Cell 28(7):1510-1520, 2016). Current crop legume plant transformation and regeneration methods still face challenges, however. For example, current methods require a substantial level of technical skill for tissue preparation, such as cutting imbibed seeds in half, precisely removing the embryo axis and primary shoot, or isolating immature embryos.

In addition, legume crop species are generally recalcitrant to genetic transformation. Even when a species such as soybean is transformable, the transformation efficiency is low, regeneration is poor, and these qualities are genotype-dependent such that only few lines with transformation and regeneration capacity have been identified. Often, the transformable lines are not elite and transformed plants therefore cannot be directly used for modern plant improvement. Current methods also require prolonged tissue culture timelines that include repeated subculture. Moreover, different legume species require different tissues (e.g., cotyledonary node, shoot meristem, callus, excised embryo for mature seed) for transformation and regeneration.

Rhizobium rhizogenes can be used to induce transformed hairy roots at high efficiency in almost any crop legume of interest, and is effective in a genotype-independent manner. However, hairy roots are a tissue that cannot be regenerated into reproductive tissues in most legume species, and thus these transformation events are a genetic dead end.

### SUMMARY

This document is based, at least in part, on the development of systems and methods for using a hairy root-like system, which is not species and genotype-dependent, to transform whole plants. The systems and methods described herein are highly desirable for the legume research and crop improvement communities. For example, the systems and methods provided herein can allow the introduction of transgenes and/or gene edits into elite lines, where they can be directly incorporated into elite breeding or research materials because they are genetically transmissible to subsequent generations.

In general, the systems and methods described herein include the use of (1) developmental regulators (DRs) that are delivered to (2) non-meristematic plant tissues (for example, cotyledons) using (3) a R. rhizogenes strain such as 18r12, K599, A4, R1000, R1200, R13333, R15834, R1601, or LBA9402. Without the DRs, these methods would result in transgenic hairy root development. However, because DRs are included, the methods provided herein result in shoots that can be regenerated into whole plants that transmit the transgenes and/or edited genes to the next generation and generations thereafter. Importantly, these methods can overcome the species and genotype-dependent limitations of current legume whole-plant transformation methods, enabling novel crop improvement strategies for these species.

In a first aspect, this document features a method for generating plant tissue having one or more genetic modifications of interest. The method can include (a) using Rhizobium rhizogenes strain (e.g., 18r12) to introduce into non-meristematic tissue of a leguminous plant (i) nucleic acid encoding one or more developmental regulators and (ii) nucleic acid including one or more sequences that, when expressed, modify cells within the plant to achieve the one or more genetic modifications of interest, wherein expression of the one or more developmental regulators induces shoot formation from the non-meristematic tissue; and (b) culturing the shoot induced by the one or more developmental regulators, to obtain modified plant tissue having the one or more genetic modifications of interest. The one or more developmental regulators can include one or more of isopentenyl transferase (IPT), BabyBoom (BBM), Shoot Meristemless (STM), Leafy Cotyledon (LEC), Wuschel (WUS), WUS homeobox-containing (Wox), and an APETALA2/Ethylene Responsive Factor (AP2/ERF) factor such as Enhancer of Shoot Regeneration (ESR1) and wound induced gene (WIND1). In some cases, the one or more developmental regulators can include BBM and WUS, WUS and IPT, WUS and STM, WUS and LEC, or WUS and ESR1 and WIND1. The non-meristematic tissue can include a cotyledon or a portion thereof. The method can include introducing nucleic acid encoding two or more developmental regulators, where the two or more developmental regulators are encoded by one T-DNA, or where the two or more developmental regulators are encoded by separate T-DNAs. The leguminous plant can be selected from the group consisting of common beans, soybeans, peas, chickpea, cowpea, pigeon pea, peanut, ground nuts, lentil, green gram, and black gram. The one or more genetic modifications can include insertion of a transgene that, when expressed, edits the plant cell DNA. The nucleic acid that modifies a plant cell can encode a targeted endonuclease (e.g., a meganuclease, zinc finger nuclease, transcription activator-like effector nuclease, or Clustered Regularly-Interspaced Short Palindromic Repeats-associated nuclease). The nucleic acid that modifies a plant cell can encode a targeted enzyme that modifies plant DNA (e.g., a cytosine deaminase or an adenosine deaminase, such as BE3 or ABE). The method can further include assaying shoot tissue induced by the one or more developmental regulators for the one or more genetic modifications of interest. The method can further include placing the shoot induced by the one or more developmental regulators into culture and inducing the shoot in culture to form a plant.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C are plasmid maps of T-DNA vectors that contain developmental regulators. Specifically, FIG. 1A is a plasmid map of a BBM vector, FIG. 1B is a plasmid map of a WUS vector, and FIG. 1C is a plasmid map of a BBM-2A-WUS vector.

FIG. 2 is an image showing shoot meristem structures formed on soybean cotyledons infected by the BBM and Wus mixed strains.

FIGS. 3A-3F are images of shoots regenerated from soybean cotyledons that were infected by BBM-2A-Wus and the mixed BBM/Wus strains. Transformation groups are indicated in parentheses. FIG. 3A shows a shoot from the BBM-2A-Wus group, while FIGS. 3B-3F show shoots from the BBM/WUS group.

FIG. 4 is a series of images showing a transformation pipeline using soybean cotyledons. The different stages of development are progressively shown in the clockwise direction. The stages include callus/cell cluster formation at wounding sites, followed by shoot meristem structure formation, de novo shoot initiation, shoot elongation and rooting.

FIGS. 5A-5C are images of a gel showing PCR analysis of regenerated shoots, testing for the presence of BBM and WUS transgenes. DNA was sampled from the plants shown in FIGS. 3A-3F. All six putatively transgenic plants showed evidence for the BBM (FIG. 5A) and Wus (FIG. 5B) genes, consistent with successful transformation of these individuals. M, DNA ladder; wc, water control; PC, plasmid DNA control; W82, non-transformed plant from genotype “Williams 82.” A soybean P450 gene was used as an internal control (FIG. 5C).

FIGS. 6A and 6B are amino acid sequence comparisons of the BBM and Wus proteins in maize and soybean. In particular, FIG. 6A shows a sequence alignment and similarity scores between a representative maize (Zm) BBM protein (SEQ ID NO:18) and a representative soybean (Gm) BBM protein (SEQ ID NO:19). FIG. 6B shows a sequence alignment and similarity scores between a representative soybean (Gm) Wus protein (SEQ ID NO:20) and a representative maize (Zm) Wus protein (SEQ ID NO:21).

FIG. 7 is an image showing regenerated plants induced by the BBM and Wus genes. The plants were transferred to soil and grown in greenhouse to set seeds.

FIGS. 8A-8C include images showing CRISPR target sites for the soybean PDS1 and PDS2 genes, and the gel pictures showing the presence of CRISPR-induced mutations in both genes from the regenerated roots. FIG. 8A is a schematic representation of the PDS1 and PDS2 genes. The gray boxes represent exons, while the white boxes represent non-coding regions. CRISPR target sites are indicated by the triangles, with the 20 bp sequence (SEQ ID NO:22) below the schematic. The restriction enzyme (SspI) site overlapping the target site is underlined. PCR amplicons of the PDS 1 (FIG. 8B) and PDS2 (FIG. 8B) genes from 4 individual regenerated roots were digested with SspI. The presence of CRISPR-induced mutations was indicated by the detection of undigested PCR products (boxed). A wild type soybean sample was used as the control. “M” indicates a DNA marker lane.

### DETAILED DESCRIPTION

Provided herein are methods and materials that can be used to achieve robust, efficient, expedited and genotype-independent genetic transformation and gene editing of leguminous plants. In some embodiments of the methods described herein, a combination of developmental (e.g., transcriptional) regulators involved in embryonic or meristematic tissue formation (Lowes et al., The Plant Cell 28(9):1998-2015, 2016) can be delivered into non-meristematic plant tissues, such as cotyledon, hypocotyledon, leaf, or root tissue, through an engineered Rhizobium rhizogenes strain to facilitate shoot regeneration. R. rhizogenes, also known as Agrobacterium rhizogenes, are soil-borne bacteria that can infect a broad range of plant species. During infection, the bacteria deliver DNA sequences, known as transfer DNAs (T-DNAs) into plant cells. T-DNAs contain genes capable of inducing de novo root formation—the phenomenon known as hairy roots. The T-DNA of R. rhizogenes strains such as 18r12 does not include the root-inducing gene (Veena and Taylor, In Vitro Cell Dev Biol Plant 43:383-403, 2007). Thus, this strain can infect plant cells and deliver T-DNA, but does not cause hairy root formation.

According to the methods provided herein, R. rhizogenes strains (e.g., 18r12) can be engineered by cloning one or more DRs into its T-DNA, such that the T-DNA can induce de novo shoot formation from infected plant tissues that otherwise would not form shoots. Expression of these DRs can be driven by plant expression promoters, such as 35S, Nos, plant tissue-specific promoters (e.g., GmLTP3-1, GmLTP3-2, GmLTP3-3, and WIND1), or inducible promoters.

In general, “developmental regulators” are agents that can direct or influence plant development, and may guide the differentiation of plant cells, organs, or tissues. In some cases, the DRs used in the methods provided herein can be transcription factors (e.g., Shoot Meristemless or Wuschel) that can stimulate plant hormone biosynthesis or plant susceptibility to/sensing of hormones that affect plant development. In some cases, the DRs used in the methods provided herein can be enzymes (e.g., IPT) that lead to increased levels of plant hormones such as cytokinins. Nucleic acids encoding DRs also are considered to be DRs for the purposes of this document, since the nucleic acid can be delivered to plant cells in order to increase the level of the encoded DR. The DR coding sequence can be operably linked to a promoter (e.g., Nos, 35S, or any other suitable promoter) that drives expression of the DR in plant cells.

This document therefor provides nucleic acid molecules containing sequences encoding one or more (e.g., one, two, three, four, or more than four) DR polypeptides, where the coding sequence(s) is operably linked to a plant expression promoter.

The terms “nucleic acid” and “polynucleotide” can be used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

In some cases, isolated nucleic acids also can be obtained by mutagenesis. For example, a naturally occurring nucleic acid sequence can be mutated using standard techniques, including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992.

Recombinant nucleic acid constructs (e.g., vectors such as T-DNA plasmids) containing sequences encoding DR polypeptides under the control of plant expression promoters also are provided herein. A “vector” is a replicon, such as a plasmid, phage, or cosmid, into which another DNA segment may be inserted so as to bring about the replication of the inserted segment. In general, vector backbones include, for example, plasmids, viruses, artificial chromosomes, bacterial artificial chromosomes (BACs), yeast artificial chromosomes (YACs), and phage artificial chromosomes (PACs), as well as RNA vectors, and linear or circular DNA or RNA molecules that include chromosomal, non-chromosomal, semi-synthetic, or synthetic nucleic acids. Vectors include those capable of autonomous replication (episomal vectors) and/or expression of nucleic acids to which they are linked (expression vectors). Generally, a vector is capable of replication when associated with the proper control elements. The term “vector” includes cloning and expression vectors, as well as viral vectors and integrating vectors. An “expression vector” is a vector that includes one or more expression control sequences to control and regulate the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available.

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the plant cell, plant, or plant tissue in which it is desired to express a DR and/or a genome editing agent.

A promoter is an expression control sequence composed of a region of a DNA molecule, typically (but not always) within 100 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically includes at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. Alternatively, constitutive promoters can promote transcription of an operably linked nucleic acid in essentially any tissue of an organism. Other classes of promoters include, without limitation, inducible promoters that confer transcription in response to external stimuli such as chemical agents, developmental stimuli, or environmental stimuli.

Any suitable DR or combination of DRs can be used in the constructs and methods provided herein to promote shoot regeneration. These include, without limitation, isopentenyl transferase (IPT), BabyBoom (BBM), Shoot Meristemless (STM), Leafy Cotyledon (LEC), Wuschel (WUS), WUS homeobox-containing (Wox), and the APETALA2/Ethylene Responsive Factor (AP2/ERF) family of factors that includes Enhancer of Shoot Regeneration (ESR1) and wound induced gene (WIND1). Any appropriate DR or combination of DRs can be delivered. In some cases, for example, a combination of DRs that includes BBM and WUS, WUS and IPT, WUS and STM, WUS and LEC, or WUS and ESR1 and WIND1 can be delivered to a plant, plant part, or plant cells. When two or more DRs are used, they can be delivered via a single T-DNA or via separate T-DNAs. In some cases, when separate T-DNAs are used to deliver two or more DRs, separate cultures of R. rhizogenes can be mixed together prior to transformation of a plant, plant part, or plant cells, or two or more T-DNAs or RNPs (e.g., CRISPR/Cas9 ribonucleoprotein complexes assembled in vitro; see, Banakar et al., Sci Rep 9:19902, 2019) can be co-bombarded into the plant, plant part, or plant cells.

In some cases, nucleotide sequences encoding such DRs can be delivered via T-DNAs to cells of leguminous plants to induce shoot generation. Expression of the delivered DR(s) can be controlled by, for example, tissue-specific promoters (e.g., cotyledon-specific promoters) or inducible promoters (e.g., wound-inducible or estradiol-inducible promoters) to direct appropriate plant cell reprogramming, differentiation, and shoot regeneration.

Non-limiting, representative sequences for at least some of the above-referenced promoters and DRs are provided below. It is to be noted, however, that homologs of these promoters and DRs exist in numerous plant species, and the methods provided herein are not limited to use of the listed promoters and DRs or to promoters and DRs having 100% identity to the provided sequences. Thus, in some cases, a promoter can have at least 80% (e.g., at least 85%, at least 90%, at least 95%, or at least 98%, or at least 99%) sequence identity to the 35S promoter sequence set forth in SEQ ID NO:1, the Nos promoter sequence set forth in SEQ ID NO:2, the LTP3 promoter sequence set forth in SEQ ID NO:3, SEQ ID NO:4, or SEQ ID NO:5, or the ESR promoter sequence set forth in SEQ ID NO:6. Further, in some cases, a DR coding sequence can have at least 80% (e.g., at least 85%, at least 90%, at least 95%, at least 98%, or at least 99%) identity to the WUS sequence set forth in SEQ ID NO:7 or SEQ ID NO:13, the STM sequence set forth in SEQ ID NO:8, the BBM sequence set forth in SEQ ID NO:9, SEQ ID NO:10, or SEQ ID NO:14, the IPT sequence set forth in SEQ ID NO:11, the LEC1 sequence set forth in SEQ ID NO:12, the WIND1 sequence set forth in SEQ ID NO:15, the WOX13LA sequence set forth in SEQ ID NO:16, or the ESR1 sequence set forth in SEQ ID NO:17.

SEQ ID NO: 1: Cauliflower mosaic virus 35S promoter
AGATTTGCCTTTTCAATTTCAGAAAGAATGCTAACCCACAGATGGTTAGAGAGGCTTACGCAGCAGGTATCATCAAGACGAT
CTACCCGAGCAATAATCTCCAGGAAATCAAATACCTTCCCAAGAAGGTTAAAGATGCAGTCAAAAGATTCAGGACTAACTGC
ATCAAGAACACAGAGAAAGATATATTTCTCAAGATCAGAAGTACTATTCCAGTATGGACGATTCAAGGCTTGCTTCACAAAC
CAAGGCAAGTAATAGAGATTGGAGTCTCTAAAAAGGTAGTTCCCACTGAATCAAAGGCCATGGAGTCAAAGATTCAAATAGA
GGACCTAACAGAACTCGCCGTAAAGACTGGCGAACAGTTCATACAGAGTCTCTTACGACTCAATGACAAGAAGAAAATCTTC
GTCAACATGGTGGAGCACGACACACTTGTCTACTCCAAAAATATCAAAGATACAGTCTCAGAAGACCAAAGGGCAATTGAGA
CTTTTCAACAAAGGGTAATATCCGGAAACCTCCTCGGATTCCATTGCCCAGCTATCTGTCACTTTATTGTGAAGATAGTGGA
AAAGGAAGGTGGCTCCTACAAATGCCATCATTGCGATAAAGGAAAGGCCATCGTTGAAGATGCCTCTGCCGACAGTGGTCCC
AAAGATGGACCCCCACCCACGAGGAGCATCGTGGAAAAAGAAGACGTTCCAACCACGTCTTCAAAGCAAGTGGATTGATGTG
ATATCTCCACTGACGTAAGGGATGACGCACAATCCCACTATCCTTCGCAAGACCCTTCCTCTATATAAGGAAGTTCATTTCA
TTTGGAGAGAACACGGGGGACT
SEQ ID NO: 2: Agrobacterium tumefaciens Nos promoter
GATCATGAGCGGAGAATTAAGGGAGTCACGTTATGACCCCCGCCGATGACGCGGGACAAGCCGTTTTACGTTTGGAACTGAC
AGAACCGCAACGTTGAAGGAGCCACTCAGCCGCGGGTTTCTGGAGTTTAATGAGCTAAGCACATACGTCAGAAACCATTATT
GCGCGTTCAAAAGTCGCCTAAGGTCACTATCAGCTAGCAAATATTTCTTGTCAAAAATGCTCCACTGACGTTCCATAAATTC
CCCTCGGTATCCAATTAGAGTCTCATATTCACTCTCAATCCAAATAATCTGCACCGTA
SEQ ID NO: 3: Soybean tissue specific GmLTP3-1 promoter
AAAATTATTTATTTATATGTTAATAAAATTATTTAACTTTTAAGAAAACTAATACAAGACGAAGAAAAATTCTTCCACCACA
TTTTTCTCTATCTCATTTTTTAAATAAATATTAAAAATATAGTATTTTCTTCCTATATTTCCTCTGATTTCATTCTTATTTA
TGTATGTTAAATTCTTATTTTTTCATTCATCCGTATAAATCAAACTCACATATGAATAGGTTTTAATATTTCACGCACACAC
ACTGTTGAACCAACCGAGCTAGACCTTTTCATTACTCTTATTTTTTTTGTTGAATTAATTAAAAAAAAAAAAGCAAAACTGA
TAGAAAGTTTCATAAATCAAGTACAAAACAGATTATGTTATTCATCTACACAATACAATCTTGAAAGAAGTTTGCGGGAAAG
ATGAGACTGGTGACAATTCATGACTGAAGCATGCTGGTTGTTTACTGATTACAATCAATTAGTATATGGGGTTGTAATATTT
ATTGTATTGTACCGAATTTCAGCACATCATGTGTTGGGTTTTGCATCTCCTAATTCAATAAAATTAGTTTGCCTTCAATAAA
GCAAAAGTGATGGAGTCTGCGAGCTAAAAGTTTGCATCTAAGACAAATTTGTAAGTTTAAGTATTATTATATTATAGATGAG
TTAAAGTAGTATTTGATGTAATTATTTTAATTTTTCTTACTTTGAAAAGAAGTTTGATTAAATACATTATCTTGAAAACACT
AAAAAAAATTACAGATTAAAAATAATTTTAAGGAGAAACTAGATGAAGTGAAGTTAGCTAGAGGATAAAAACTTTTTCCCTC
ACTCTGTTCCCCACACTGATCTTTCTTTTGTCTCTGCACTTTCTCTTTTTTTTTTCTATCTCCTTACTCTCTCTCTCTCTCT
CCCCACTTTTTACTGAGTTTCTTTTTAAAGTTAATAGAGAAAATAAATTTTATAAAGGATAACGATACTCCTGGTCTGCAGT
TCATTTTTGGGTAGATAAAATGGATGCAAGAGAATTATAGAAAGACAAGCAAGGAAGAAAATTTTATTTATGGGCAGATAAA
ATTGATGGAAGAGAATGATAGAAAGGAAGAAAAAAAATATCGACTATGGAAGGATGGTTTTAACAACACTTTTTTTCTAACA
TTTTTTTTATTGACTAAAATTTATTATAAATTACACATGTTTTAAAATCTTACTTTTAAATCAAGAGGGACATATAGTCATT
AAGCAAGAATGAGACCTATTAAAATTTATAATTTTTAATAAATTTTAACTAATAATAAAATAGAGAGTGTATATTAAAAAGA
AAGTAGAGGTTACAAAGAATAATTTTACTACCATTTCTCTCAAAGTCTCAACGTCACTAACGGATTTACATGGAAATTAATG
GGGTAATTGTTCTTACAAACTAATTTTTTTACTTAAATATATTAAATAAATATTTTGTTGTCATCATAAAAAAAATAGATTT
TGTCTTCATAAGATAATTTTTTAATAAGATGAATGTAAATAGTTGTAAAAGATAAAAGAGAAAATAAATTAATATTAATTTT
ATCACTTTATTCTTTTAAATCTTACTGTTTTTAATTTTTTTTTATTACCATTTTTAATAAATCTTATATATTTTTTTGTTTG
AAAAATATTGGACTATTTTTGTCCTACTAAAAAATTTCTCAATCCGCCGGTGCTCAAAATGAAATATGTTACCGACTTCTTG
TTCATTAGAATGTAGAGTCTGGAAGATAGATAAACCAGGGGCTGCATGGAAAATGGGTACAGTATATATATATATATATGTA
AATTAATGCTGCTTCAATTAATTACTTACTATACCTTCCTTTTGAAATCTAGACACTAGACATGGTTATGGTTACCCTTCAC
CTCTTGCCTCAGAGTAGAGTAATTGTTGCATTTAACTAACCCAACCACAATCCATTATTACACTGAATTCATCTCAACTACC
CAATAATAAGCACCACCACATGCCCCTTCTGCAATTGCACTGCACCCCATAGACACATAGCCCCCCTTCTCCCATATATAAA
CCCAAACCCACCACAGTGTCTCAAAACACACCACACCTTCAAACCCTAACCCTAGAAGAAGAAAAAAACCACAACCACCACC
ATCTAGGGTTTTTCTTTTTCTC
SEQ ID NO: 4: Soybean tissue specific GmLTP3-2 promoter
GATTTTGTGTGTCAAAGAAAGTATGCTCATGAAATTCTAGGAAGGTTTGAAATGGATAAAAGCAATCCAGTGAAGAATCCTA
TTGTTCCAGGCAGGAAATTGTCAAAGGATGAAGCAGGAACCAAAGTTGATGAAACTTTGTTCAAACAGGTAGTTGGCAGTCT
GATGTACTTGACTGCAACTCAACCTGATTTGATGTATGGAGTGAGTCTTATTAGTAGATTCATGTCTTGCCCAACTGAATCT
CATTGGCTTGCAGCAAAAAGAATATTAAGGTATTTGAAAGGTACAACTGAGCTTGGAATTTTCTACAAAAAGGATGGTTGCA
CAAATTTGGTGGCTTACACCAACAGTGATTTTGTTGGTGATTTGGATGATCGTAGGAGCACTTCTGGCTTTGCGTTCTTACT
TGGTTCTGGAGCAGTTTCATGGTCTTCTAAAAAACAACCTATAGTAACATTGTCTACTACTGAGGCTGAGTACATAGCTGCT
GCGTTTTGTGTCTGTCAGTGTATTTGGTTGAGAAGAGTTTTAGAGAAACTTGGTCATAAGGAGGAAAATAGTACTTTGATTC
AATGTGACAATAATTCTACTATACAACTATCAAAGAATTTTGTTTTTCATGGCAGAAGCAAACATATTAATACAAGGTTTCA
TTTTCTTAGAGATTTGACCAGAGACAAAATTGTGGAGTTGAGTTACTGTAATTCTCAAGAACAGGTTGCAGACATAATGACA
AAACCTCTCAAGTTAGAACAATTCTTGAAATTACGGAGTATGTTGGGAATGGTTGATGTGTCAGTTATAAACTAAATGTTTG
TTTTTCTTTTTAGTTTAAGGAAGGGAATGTTGACGTGTAAATAGAAGTTAATAGAAGTTAAGAAGTTAGCGGTAGTTTTTTG
TAAATAGGAGTTATGTGGAGTAGTTATATGGAGTAGTTATGTGGAGTAGTTATAAATATATTGTAACTGCAGTGGTCTATAT
ATTGTACCTTTGTAACTATAGTAATTGAATTTGAAGATTAAAAAAAAGAGCATTCTCTCTAAAACTTGTATGTCATTGTTCT
TTTTAACAATAATATACTAGTACGTACTTAAACTTACAATCTCTAAAAAAAAATACTTAAACTTACAATTTGTTTTAGAAGC
AAACTTAACTCACAAGCTCCATGCCTTCATGATGAAATTATTTTAATTTTTCTTTCTTTGAAAAGAGGTTGGATTAAATACG
TTATCTTGAAAACTCTAAAAAAAATTAATCAGAATAATCTTTATATATAAAAGAAAAAAAATAATTAATTTTAAAAAGAAAC
TAGATGAAGTGAAGTTAGAGGATAGATTTTTTTCCCTCACTCTGTTCCCACTCTTTCTTTTGTCTCTACACTTTCTCTTTTT
TTATGTCTCCTAACTCGATCTCCATTTTTTACTGAGTTTCTTTTTTAAGTTAAAAGAGAAAATAAATATTTTTATAGAATCT
CTATCCATTTCATCTACTCCTGCACTTCAGTTTTGGGCAGATGAAATGGATGAAAAGAGAGTTATAGAAAGAAAAGAAAAGG
AAGAAAAATTATTTTTGGGAAGATAAAATGGATGGAAGGTTGTTGTTCATCAGAATCTGGAGTCTGGAAGATAGATAAACCC
GGGGCTGCATGGAAAATGAGTAAAGTGATTTACTTCTTGACACGTAACACTGATATATATATATATATATATATATATATAT
ATATATATATATATATATATATATATTCCAGTTCAGTACACATAAATATCAATGTTGCTTCAATGAATTACTACAACTTCCT
TTGAAATCTAGACACATGGTTATGGTTACCCTTCACTTCTTGCCTCAGAGTATAGTAATTATAGCATTTAACTTACCCAACC
ACAATCCATTATTACACTGAATTCATCTCAACTACCCAAATAAGCACCACCCACATGCCCCTTCTGCCTGCACTGCACCCCA
TAGACACATAGTCCCATAGCCTTCTCCCAATATATAAGCCCAACCCTAAACCCACCACATAGTAGTTTCTCAAAACACATCA
CACCTTCAAACCCGAACCCTAACCCTAGAAGAAAGAAAAAAACCCACAACACACAATCAGTACTACCAACT
SEQ ID NO: 5: Soybean tissue specific GmLTP3-3 promoter
TGGAGACAACACACAGAAACACCAGACAGTAAGGTTTTCATAGGCACCGAAAAGCGTGAAAAGTGTGCAAAATCATAAGCTA
GCCTGTTCACAATTGACTTGTGCGTCACTAGTTTTGCTAAACACTGACCAGCTCATACCAATTTGAATACATTTGTGATCAA
ATAAGTGGCTCAAACATGATAGGTGACCGATTTTCATTCAAACCGATTGAATCGGTCGATGAAAATGGAGCATGATCAATAA
CACTTGGCGAAAGGAGTAAGAGGCCTTACGAGTGAAGTGGTGAAAGAGAATCTTTCTTAGAGCACCAGGATGAGAAAGGATG
GTATAAATGTTAACACGTTACAATTTGTTTACGACACAATTTTCTGTTGCCTAACCTTGAGTGGGTAATGTCATCCTCCCCA
AAGTAAATCAGATTCCTTTTTTTTTTTTCTAAAATAAAGATATTTTGTTCACCACACATAATTTTACGTTTTTGATAAATTG
TAGAGATATTATGTTCATCCAGAAACTATACAATAACTTCATTATTCTTCTCTTTTGTTATCATATCACTTGTTATTTTATA
TTTTTTCTTTTTTTGTTTTCTATTTTTTCTATCTTTCTTATTTATAAAAAAATAATACACATAAAATAAAATTTACATCATT
TTTGCATCTATTTCTCTCTATATCATTTATTATATATTTTTTCTTTCCTCTCCATTGGTTGTTTTTTTCAAAAACCAAAACT
TCTTTCTCTGCTTGCTAGTCTTAAAAATTATTGTATAATTTAGGATAAATAGTCATTTTTGTCTTTAAATGTGTAATTCGCT
CACAAATGTGTCCCTGAAAGATAAAAATATAAAATTTAGTCCTTGAAAATGTAAAACATGCAACAAGTATATCCGATCATTA
ACTTCCGTCTGGTACCGTTAATAAAATAACTTATATGACACATAGGGACGAATTGTCACTAACATGAATGATTGTCAATGTG
GTCATCTCTATTTGTCAGCATAGGGACATATTTGTCATATAATATTTTTTTTTTACTTTTTGTCTTCTCATTTTGCTGACAA
ATGTGTCCCTAAGAGATAAAAATACAAAATTTAGTTCCCGAAAGTGAAAAAAAGATGACAAATATATCCGGACGTTAATAGT
AGATTGTGATTAATTATTATAATTTAAAAAAATTTATAATGATTGCAACAAAATTCTGGGAGAGCTAAATCATATTGGTAAG
TTTTTGTTTCCATTATAAAAATTTTAAGCGAAGTAAAAAAAATTACTATAAAACAATAAAAAAATCATGATTTTTGTTTAAT
CAAACACTATTTATTTAATTATTTTTTTATAGATTTTTCATAATAAAATATGAATCTCTATTAGTATATGCAATCACATCAA
ATTATAAATTATAATAAAAGTTTTTTTATTAACTAATCATTTAAAAAATCATAACTTTAAAAAAATAATTCCAAAGGAGGTG
AGTATTTTTGTACAAATTAAAAGTGTCAATGTAATTGCAATGTTTCCTAATTCGATCTTTTTATCGTCATCAGTATAAAAAA
TTTTAAATTATAATAATTATTCACAATCTATTATTAATATCTAGATATATTTATCAGATTTTTTTCACTTTTAATGAATAAA
TTTTGTATATTAATATTTTAAGAATGTATTTATCAATGACTTTTACAAGTAACTATTTATTACTATTATATAAGTATATTTA
TCTAAAAAAATGTATAAAAACATTACATATCAATGAAGAAACTAGTTATTGCAGGACCAATTAATAAAAGAAATACAATTCA
ATCCTCGAAATCTGTCAAGGTATGGAGGGATAAGCTATAATATTAAATATATCATCCTATTCAGTCGTATTTGGGGCACCAC
CAAATCAAGCGTCACATATTAAGCCAGGTGCCAAAAGATTATACTATGCATGCACCACCACCTAGCATTAAATTGAGCACAA
CTGCCACCAAACAAAAAACAGTACTGCCACATGCATTTTCCATAACCCTTAGTTACCTTCCCACAGCCCTAGGAGTCCTATC
TTATCTTCTATATAACTCCCCACACTTGCACCACTTCACTCACCAAACAATAAGCAAGAATTTCCATTGCATGCAAAGCAAA
TACGCATTATATTACTAGTTTTAATTTGTTTTGTCGTTTGCTTCTTTCTTTGATTA
SEQ ID NO: 6: Arabidopsis thaliana tissue specific AtESR1 promoter
CGCAAACGCAGCCATTACAACGCTATTTCAAAACTTATTTAAACAATTAGTAATACTTACCAAAGCTTTTGAAAGATACTCC
AGTAAACTTGTCAAATTCTTATATTGTTCTAATAGACTTTATAATAAAACGTAAGCATCGGGCGATATGGCATGATTATTTC
ATGACAGAAGAAATAAATAATTTTAACAAAAAAAAAGACAGAAGCAATAAATAAAAAACAAGCTTCTGATACATGAAATACA
TATATGTCTCATACATACGTTTAGACAAACCTGAAATGTCCTCTTCGTACAATAATATCCACAAGTGTCAGATTTACACTGA
AGTGTTGCTATGCATATCTTTTGTTCCATACTTTTCTTACAAAATCATTTATTTTCTTCTCGATATCACCGATTGGTGTATG
TTGTAATCATACATTAACATTAACAAACAAAGATACTTTTTCAAGGATTATTCTAATTGGCAGTTTTAGAAATAATGGAATT
ATCCTTTACTAATCATACAGAAATAATGTAAATATGTTTAGCATGTGTGAAATGACTGTTGGTCGATTTTTAACTTTAATAA
ATAAAAAAGCAATAAGAACGTGGTTTTATTTCGCCACTCCCACTTGCATCGTCATCATCAAAGAAAAACACTAATGTCTAGA
CCAAAGATTTAAAACATCTACCCCATATATATATGATGAACAAGATAGCAAGTAAATTTAAATGTAAAATTAAATTTTAGTT
TGCTAAGATTAATATACAAAAGAAGTATTATCAATTTATCAGTTATTAATCAAATCAAGTTTTAAGTGCAACTCAAAAGTTT
CCATGCTTATATAGTTATTTGTATACTACTATACTGTATGTGCAAGAAAAGCATTTATACTCTTCGCCATATATTTCAAACT
TCACAAAATTTAATTAAATTTTTATACCATTTATGTACCTATAAATAGATAGAAGAAGCTCCATCTCTTTCAAACTATCAAC
CACCAAAATCTTTCACATTACACCTTCCTTTTGTCCTCAAACCAAAACCCTAGAAACCAAAA
SEQ ID NO: 7: Zea maize WUS2
ATGGCGGCCAATGCGGGCGGCGGTGGAGCGGGAGGAGGCAGCGGCAGCGGCAGCGTGGCTGCGCCGGCGGTGTGCCGCCCCA
GCGGCTCGCGGTGGACGCCGACGCCGGAGCAGATCAGGATGCTGAAGGAGCTGTACTACGGCTGCGGCATCCGGTCGCCCAG
CTCGGAGCAGATCCAGCGCATCACCGCCATGCTGCGGCAGCACGGCAAGATCGAGGGCAAGAACGTCTTCTACTGGTTCCAG
AACCACAAGGCCCGCGAGCGCCAGAAGCGCCGCCTCACCAGCCTCGACGTGAACGTGCCCGCCGCCGGCGCGGCCGACGCCA
CCACCAGCCAACTCGGCGTCCTCTCGCTGTCGTCGCCGCCGCCTTCAGGCGCGGCGCCTCCCTCGCCCACCCTCGGCTTCTA
CGCCGCCGGCAATGGCGGCGGATCGGCTGTGCTGCTGGACACGAGTTCCGACTGGGGCAGCAGCGGCGCTGCGATGGCCACC
GAGACATGCTTCCTCCAGGACTACATGGGCGTGACGGACACGGGCAGCTCGTCGCAGTGGCCACGCTTCTCGTCGTCGGACA
CGATAATGGCGGCGGCCGCGGCGCGGGCGGCGACGACGCGGGCGCCCGAGACTCTCCCTCTCTTCCCGACCTGCGGCGACGA
CGGCGGCAGCGGTAGCAGCAGCTACTTGCCGTTCTGGGGTGCCGCGTCCACAACTGCCGGCGCCACTTCTTCCGTTGCGATC
CAGCAGCAACACCAGCTGCAGGAGCAGTACAGCTTTTACAGCAACAGCAACAGCACCCAGCTGGCCGGCACCGGCAACCAAG
ACGTATCGGCAACAGCAGCAGCAGCCGCCGCCCTGGAGCTGAGCCTCAGCTCATGGTGCTCCCCTTACCCTGCTGCAGGGAG
TATGTGA
SEQ ID NO: 8: Arabidopsis thaliana STM
ATGGAGAGTGGTTCCAACAGCACTTCTTGTCCAATGGCTTTTGCCGGGGATAATAGTGATGGTCCGATGTGTCCTATGATGA
TGATGATGCCGCCCATCATGACATCACATCAACATCATGGTCATGATCATCAACATCAACAACAAGAACATGATGGTTATGC
ATATCAGTCACACCACCAACAAAGTAGTTCCCTTTTTCTTCAATCACTAGCTCCTCCCCAAGGAACTAAGAACAAAGTTGCT
TCTTCTTCTTCTCCTTCCTCTTGTGCTCCTGCCTATTCTCTAATGGAGATCCATCATAACGAAATCGTTGCAGGAGGAATCA
ACCCTTGCTCCTCTTCCTCTTCTTCAGCCTCTGTCAAGGCCAAGATCATGGCTCATCCTCACTACCACCGCCTCTTGGCCGC
TTATGTCAATTGTCAGAAGGTTGGAGCACCACCGGAGGTTGTGGCGAGGCTAGAGGAGGCATGCTCGTCTGCCGCAGCCGCT
GCCGCATCTATGGGACCAACAGGATGTCTAGGTGAAGATCCAGGGCTTGATCAATTCATGGAAGCTTACTGTGAAATGCTCG
TTAAGTATGAGCAAGAGCTCTCCAAACCTTTCAAGGAAGCTATGGTCTTCCTTCAACGTGTCGAGTGTCAATTCAAATCCCT
CTCTCTATCCTCACCTTCCTCTTTCTCCGGTTATGGAGAGACAGCAATTGATAGGAACAATAATGGGTCATCCGAGGAAGAA
GTCGATATGAACAATGAATTTGTAGATCCACAAGCTGAGGATAGAGAGCTTAAAGGACAGCTCTTGCGCAAGTACAGTGGTT
ACTTAGGGAGCCTCAAGCAAGAGTTCATGAAGAAGAGGAAGAAAGGAAAGCTCCCTAAAGAAGCTCGTCAACAACTGCTTGA
TTGGTGGAGCCGTCACTACAAATGGCCTTACCCTTCGGAGCAACAAAAGCTCGCCCTTGCGGAATCAACGGGGCTGGACCAG
AAACAGATAAACAATTGGTTCATAAACCAGAGGAAACGGCATTGGAAGCCGTCGGAGGACATGCAGTTTGTAGTAATGGACG
CAACACATCCTCACCATTACTTCATGGATAATGTCTTGGGCAATCCTTTCCCAATGGATCACATCTCCTCCACCATGCTTTG
A
SEQ ID NO: 9: Zea maize BBM
ATGGCCACTGTGAACAACTGGCTCGCTTTCTCCCTCTCCCCGCAGGAGCTGCCGCCCTCCCAGACGACGGACTCCACACTCA
TCTCGGCCGCCACCGCCGACCATGTCTCCGGCGATGTCTGCTTCAACATCCCCCAAGATTGGAGCATGAGGGGATCAGAGCT
TTCGGCGCTCGTCGCGGAGCCGAAGCTGGAGGACTTCCTCGGCGGCATCTCCTTCTCCGAGCAGCATCACAAGGCCAACTGC
AACATGATACCCAGCACTAGCAGCACAGTTTGCTACGCCAGCTCAGGTGCTAGCACCGGCTACCATCACCAGCTGTACCACC
AGCCCACCAGCTCAGCGCTCCACTTCGCGGACTCCGTAATGGTGGCCTCCTCGGCCGGTGTCCACGACGGCGGTGCCATGCT
CAGCGCGGCCGCCGCTAACGGTGTCGCTGGCGCTGCCAGTGCCAACGGCGGCGGCATCGGGCTGTCCATGATTAAGAACTGG
CTGCGGAGCCAACCGGCGCCCATGCAGCCGAGGGTGGCGGCGGCTGAGGGCGCGCAGGGGCTCTCTTTGTCCATGAACATGG
CGGGGACGACCCAAGGCGCTGCTGGCATGCCACTTCTCGCTGGAGAGCGCGCACGGGCGCCCGAGAGTGTATCCACGTCAGC
ACAGGGTGGAGCCGTCGTCGTCACGGCGCCGAAGGAGGATAGCGGTGGCAGCGGTGTTGCCGGCGCTCTAGTAGCCGTGAGC
ACGGACACGGGTGGCAGCGGCGGCGCGTCGGCTGACAACACGGCAAGGAAGACGGTGGACACGTTCGGGCAGCGCACGTCGA
TTTACCGTGGCGTGACAAGGCATAGATGGACTGGGAGATATGAGGCACATCTTTGGGATAACAGTTGCAGAAGGGAAGGGCA
AACTCGTAAGGGTCGTCAAGTCTATTTAGGTGGCTATGATAAAGAGGAGAAAGCTGCTAGGGCTTATGATCTTGCTGCTCTG
AAGTACTGGGGTGCCACAACAACAACAAATTTTCCAGTGAGTAACTACGAAAAGGAGCTGGAGGACATGAAGCACATGACAA
GGCAGGAGTTTGTAGCGCCTCTGAGAAGGAAGTCCAGTGGTTTCTCCAGAGGTGCATCCATTTACAGGGGAGTGACTAGGCA
TCACCAACATGGAAGATGGCAAGCACGGATTGGACGAGTTGCAGGGAACAAGGATCTTTACTTGGGCACCTTCAGCACCCAG
GAGGAGGCAGCGGAGGCGTACGACATCGCGGCGATCAAGTTCCGCGGCCTCAACGCCGTCACCAACTTCGACATGAGCCGCT
ACGACGTGAAGTCCATCCTGGACAGCAGCGCCCTCCCCATCGGCAGCGCCGCCAAGCGCCTCAAGGAGGCCGAGGCCGCAGC
GTCCGCGCAGCACCACCATGCGGGTGTCGTTTCCTATGACGTTGGGAGGATTGCCAGCCAACTGGGAGATGGCGGTGCCCTC
GCTGCGGCCTATGGTGCTCACTATCACGGTGCCGCGTGGCCAACGATTGCATTCCAGCCGGGCGCGGCGTCCACCGGACTGT
ACCATCCTTACGCGCAGCAGCCTATGCGCGGCGGTGGATGGTGTAAACAAGAGCAAGATCACGCTGTGATAGCAGCGGCACA
CTCCTTGCAGGATCTTCATCATTTGAATCTCGGAGCCGCCGGGGCCCACGACTTTTTCTCGGCAGGGCAGCAGGCCGCCGCC
GCTGCGATGCACGGCCTGGGTAGCATCGACAGTGCGTCGCTGGAGCACAGCACCGGCTCCAACTCCGTCGTCTACAACGGCG
GGGTCGGCGACAGCAACGGCGCCAGCGCCGTCGGCGGCAGTGGCGGTGGCTACATGATGCCGATGAGCGCTGCCGGAGCAAC
CACTACATCGGCAATGGTGAGCCACGAGCAGGTCCATGCACGGGCCTACGACGAAGCCAAGCAGGCTGCTCAGATGGGGTAC
GAGAGCTACCTGGTGAACGCGGAGAACAATGGTGGCGGAAGGATGTCTGCATGGGGGACTGTCGTGTCTGCAGCCGCGGCGG
CAGCAGCAAGCAGCAACGACAACATGGCCGCCGACGTGGGCCACGGCGGCGCGCAGCTGTTCAGTGTCTGGAACGACACTTA
A
SEQ ID NO: 10: Arabidopsis thaliana BBM
ATGAACTCGATGAATAACTGGTTAGGCTTCTCTCTCTCTCCTCATGATCAAAATCATCACCGTACGGATGTTGACTCCTCCA
CCACCAGAACCGCCGTAGATGTTGCCGGAGGGTACTGTTTTGATCTGGCCGCTCCCTCCGATGAATCTTCTGCCGTTCAAAC
ATCTTTTCTTTCTCCTTTCGGTGTCACCCTCGAAGCTTTCACCAGAGACAATAATAGTCACTCCCGAGATTGGGACATCAAT
GGTGGTGCATGCAATAACATTAACAATAACGAACAAAATGGACCAAAGCTTGAGAATTTCCTCGGCCGCACCACCACGATTT
ACAATACCAACGAGACCGTTGTAGATGGAAATGGCGATTGTGGAGGAGGAGACGGTGGTGGTGGCGGCTCACTAGGCCTTTC
GATGATAAAAACATGGCTGAGTAATCATTCGGTTGCTAATGCTAATCATCAAGACAATGGTAACGGTGCACGAGGCTTGTCC
CTCTCTATGAATTCATCTACTAGTGATAGCAACAACTACAACAACAATGATGATGTCGTCCAAGAGAAGACTATTGTTGATG
TCGTAGAAACTACACCGAAGAAAACTATTGAGAGTTTTGGACAAAGGACGTCTATATACCGCGGTGTTACAAGGCATCGGTG
GACAGGTAGATACGAGGCACATTTATGGGACAATAGTTGCAAAAGAGAAGGCCAGACTCGCAAAGGAAGACAAGTTTATCTG
GGAGGTTATGACAAAGAAGAAAAAGCAGCTAGGGCTTACGATTTAGCCGCACTAAAGTATTGGGGAACCACCACTACTACTA
ACTTCCCCTTGAGTGAATATGAGAAAGAGGTAGAAGAGATGAAGCACATGACGAGGCAAGAGTATGTTGCCTCTCTGCGCAG
GAAAAGTAGTGGTTTCTCTCGTGGTGCATCGATTTATCGAGGAGTAACAAGGCATCACCAACATGGAAGGTGGCAAGCTAGG
ATCGGAAGAGTCGCCGGTAACAA
SEQ ID NO: 11: Agrobacterium tumefaciens IPT
ATGGATCTGCGTCTAATTTTCGGTCCAACTTGCACAGGAAAGACGTCGACCGCGATACGTCTTGCCCAGCAGACTGGCCTTC
CAGTCCTTTCGCTCGATCGGGTCCAATGCTGTCCTCAACTGTCAACCGGAAGCGGACGACCAACAGTGGAAGAACTGAAAGG
AACGACCCGTCTATACCTTGAAGATCGGCCTCTGGTGAAGGGTATCATCGCAGCCAAGCAAGCTCACGAAAGGCTGATCGGG
GAAGTGTACAATTATGAGGCCCACGGCGGGCTTATTCTTGAGGGAGGATCTATCTCGTTGCTCAGGTGCATGGCGCAAAGCA
GTTATTGGAGTACCGATTTTCGTTGGCATATTATTCGCCACAAGTTAGCAGACGAGGAGACATTCATGAACGCGGCCAAGGC
CAGAGTTAGGCAGATGTTGCGCCCTGCTGTAGGCCCATCTATTATTCAAGAGTTGGTTCATCTTTGGAATGAGCCTCGGCTG
AGGCCCATACTGAAAGAGATCGACGGATATCGATATGCCATGTTATTTGCTAGCCAGAACCAGATCACACCCGATATGCTAT
TGCAGCTTGACCCAGATATGGAGGGTGAGTTGATTCATGGAATCGCTCAGGAGTATCTCATCCATGCGCGCCGGCAGGAGCA
GGAATTCCCTCCAGTGAGCGTGGTCGCTTTCGAAGGATTCGAAGGTCCACCGTTCGGAATGTGCTAG
SEQ ID NO: 12: Glycine max GmLEC1
ATGGAAACTGGAGGCTTTCATGGCTACCGCAAGCTCCCCAACACAACCTCTGGGTTGAAGCTGTCAGTGTCAGACATGAACA
TGAACATGAGGCAGCAGCAGGTAGCATCATCAGATCAGAACTGCAGCAACCACAGTGCAGCAGGAGAGGAGAACGAATGCAC
GGTGAGGGAGCAAGACAGGTTCATGCCAATCGCTAACGTGATACGGATCATGCGCAAGATTCTCCCTCCACACGCAAAAATC
TCCGATGATGCAAAGGAGACAATCCAAGAGTGCGTGTCGGAGTACATCAGCTTCATCACCGGGGAGGCCAACGAGCGTTGCC
AGAGGGAGCAGCGCAAGACCATAACCGCAGAGGACGTGCTTTGGGCAATGAGTAAGCTTGGATTCGACGACTACATCGAACC
GTTAACCATGTACCTTCACCGCTACCGTGAGCTGGAGGGTGACCGCACCTCTATGAGGGGTGAACCGCTCGGGAAGAGGACT
GTGGAATATGCCACGCTTGCTACTGCTTTTGTGCCGCCACCCTTTCATCACCACAATGGCTACTTTGGTGCTGCCATGCCCA
TGGGGACTTACGTTAGGGAAACGCCACCAAATGCTGCGTCATCTCATCACCATCATGGAATCTCCAATGCTCATGAACCAAA
TGCTCGCTCCATATAA
SEQ ID NO: 13: Glycine max GmWUS1
ATGATGGAACCTCAACAACAACAACAACAAGCACAAGGGAGCCAACAACAACAACAAAACGAGGATGGTGGCAGTGGAAAAG
GGGGGTTTCTGAGCAGGCAAAGTAGTACACGGTGGACTCCAACAAACGACCAGATAAGAATATTGAAGGAACTTTACTACAA
CAATGGAATTAGATCCCCGAGTGCAGAGCAGATTCAGAGGATCTCTGCTAGGCTGAGGCAGTACGGTAAGATTGAAGGCAAG
AATGTCTTTTATTGGTTCCAGAACCACAAAGCTCGAGAAAGGCAGAAGAAAAGGTTCACTTCTGATCATAATCATAATAATG
TCCCCATGCAAAGACCCCCAACTAATCCTTCTGCTGCTTGGAAACCTGATCTAGCTGATCCCATTCACACCACCAAGTATTG
TAACATCTCTTCTACTGCAGGGATCTCTTCGGCATCATCTTCTGTTGAGATGGTTACTGTGGGACAGATGGGGAATTATGGG
TATGGTTCTGTGCCCATGGAGAAAAGTTTTAGGGACTGCTCGATATCAGCTGGGGGTAGCAGTGGCCATGTTGGATTAATAA
ACCACAACTTGGGGTGGGTTGGTGTGGACCCATATAATTCCTCAACCTATGCCAACTTCTTTGACAAAATAAGGCCAAGTGA
TCAAGAAACCCTTGAAGAAGAAGCAGAGAACATTGGTGCTACTAAGATTGAAACCCTCCCTTTATTCCCTATGCACGGTGAG
GACATCCATGGCTATTGCAACCTCAAGTCTAATTCGTATAACTATGATGGAAACGGCTGGTATCATACTGAAGAAGGGTTCA
AGAATGCTTCTCGTGCTTCCTTGGAGCTCAGTCTCAACTCCTACACTCGCAGGTCTCCAGATTATGCTTAA
SEQ ID NO: 14: Glycine max GmBBM1
ATGGGGTCTATGAATTTGTTAGGTTTTTCTCTCTCTCCTCACGAAGAACACCCTTCTAGTCAAGATCACTCTCAAACGACAC
CTTCTCGTTTTAGCTTCAACCCTGATGGATCAATCTCAAGCACTGATGTAGCAGGAGGCTGCTTTGATCTCACTTCTGACTC
AACTCCTCATTTACTTAACCTTCCTTCTTATGGCATATACGAAGCATTTCACAGAAACAATAGTATTAACACCACTCAAGAT
TGGAAGGAGAACTACAACAGCCAAAATTTGCTATTGGGAACTTCGTGCAATAAACAAAACATGAACCAAAACCAACAGCAAC
AGCCAAAGCTTGAAAACTTCCTCGGTGGACACTCATTTGGCGAACATGAGCAAACCTACGGTGGTAACTCAGCCTCTACAGA
TTACATGTTTCCTGCTCAGCCAGTATCGGCTGGTGGTGGTGGTAGTGGTGGTGGCAGTAACAATAACAACAACAGTAACTCC
ATAGGGTTATCCATGATAAAGACATGGTTGAGGAACCAACCACCGAACTCAGAAAACATCAACAACAACAATGAAAGTGGTG
GCAATATTAGAAGCAGTGTGCAGCAAACTCTATCACTTTCCATGAGTACTGGTTCACAATCAAGCACATCACTGCCCCTTCT
CACTGCTAGTGTGGATAATGGAGAGAGTTCTTCTGATAACAAACAACCAAACACCTCGGCTGCACTTGATTCCACCCAAACC
GGAGCCATTGAAACTGCACCCAGAAAGTCCATTGACACTTTTGGACAGAGAACTTCTATCTACCGTGGTGTAACAAGGCATA
GGTGGACGGGGAGGTACGAGGCTCACCTGTGGGATAATAGTTGTAGAAGAGAGGGACAGACTCGCAAAGGAAGGCAAGTTTA
CTTGGGTGGTTATGATAAAGAAGAAAAGGCAGCTAGAGCCTACGATTTGGCAGCACTAAAATACTGGGGAACAACCACAACA
ACAAATTTTCCAATTAGCCACTATGAGAAAGAGTTGGAAGAAATGAAGCACATGACTAGGCAAGAGTACGTTGCGTCATTGA
GAAGGAAGAGTAGTGGGTTTTCTCGCGGTGCATCCATTTATCGAGGAGTGACGAGACACCACCAACATGGAAGGTGGCAAGC
GAGGATTGGAAGAGTTGCTGGCAACAAGGATCTTTACTTGGGAACTTTTAGCACCCAAGAAGAGGCAGCGGAAGCATATGAT
GTAGCAGCAATCAAATTCCGAGGACTAAGTGCTGTTACAAACTTTGACATGAGCAGATATGACGTGAAAAGCATACTTGAGA
GCACCACTTTGCCAATAGGTGGTGCTGCAAAGCGTTTGAAGGATATGGAGCAGGTTGAACTGAGTGTGGATAATGGTCATAG
AGCAGATCAAGTAGATCATAGTATCATCATGAGTTCTCACCTAACTCAAGGAATCAATAACAACTATGCAGGAGGGGGAACA
GCAACTCATCATAACTGGCACAATGCTCATGCATTCCACCAACCTCAACCTTGCACCACCATGCACTACCCTTATGGACAAA
GAATTAATTGGTGCAAGCAAGAACAACAAGACAACTCTGATGCCCCTCACTCTTTGTCTTATTCAGATATTCATCAACTTCA
GCTAGGGAACAATGGAACACATAACTTCTTTCACACAAATTCAGGGTTGCACCCTATGTTGAGCATGGATTCTGCTTCCATT
GACAATAGCTCTTCTTCTAACTCGGTTGTTTATGATGGTTATGGAGGTGGTGGGGGCTACAATGTGATGCCTATGGGAACTA
CTACTGCTGTTGTTGCAAGTGATGGTGATCAAAATCCAAGAAGCAATCATGGTTTTGGTGATAATGAGATAAAAGCACTTGG
TTATGAAAGTGTGTATGGCTCTGCAACTGATTCTTATCATGCACATGCAAGGAACTTGTATTATCTTACTCAACAGCAATCA
TCTTCTGTTGATACAGTGAAGGCTAGTGCATATGATCAAGGGTCTGCATGCAATACTTGGGTTCCAACTGCTATTCCAACTC
ATGCACCCAGATCAACTACTAGTATGGCTCTCTGCCATGGGGCTACTACACCCTTCTCTTTATTGCATGAATAG
SEQ ID NO: 15: Arabidopsis thaliana AtWIND 1
ATGGAAAAAGCCTTGAGAAACTTCACCGAATCTACCCACTCACCAGACCCTAATCCTCTCACAAAATTCTTCACTGAACCTA
CAGCCTCACCTGTTAGCCGCAACCGCAAACTGTCTTCAAAAGATACCACTGTAACCATCGCCGGAGCTGGCAGCAGCACGAC
GAGGTACCGCGGCGTACGCCGGAGGCCGTGGGGACGATACGCGGCGGAGATACGTGACCCAATGTCGAAGGAGAGACGTTGG
CTCGGAACATTTGACACGGCGGAACAAGCCGCTTGTGCTTACGACTCTGCGGCTCGTGCCTTTCGTGGAGCAAAGGCTCGTA
CTAATTTTACTTATCCGACAGCTGTCATTATGCCTGAACCAAGGTTTTCTTTTTCCAACAAGAAATCTTCGCCGTCTGCTCG
TTGTCCTCTTCCTTCTCTACCGTTAGATTCCTCTACCCAAAACTTTTACGGTGCACCGGCAGCGCAGAGGATCTATAATACA
CAGTCTATCTTCTTACGCGACGCCTCGTGTTCCTCTCGTAAAACGACTCCGTATAATAACTCTTTCAACGGCTCATCATCTT
CTTACTCAGCATCGAAAACGGCATGCGTTTCTTATTCCGAAAACGAAAACAACGAGTCGTTTTTCCCGGAAGAATCTTCTGA
TACTGGTCTATTACAAGAGGTCGTTCAAGAGTTCTTGAAGAAAAATCGCGGCGTTCCTCCTTCTCCACCAACACCACCGCCG
GTGACTAGCCATCATGACAACTCTGGTTATTTCTCTAATCTCACTATATACTCTGAAAATATGGTTCAAGAGACTAAGGAGA
CTTTGTCGTCGAAACTAGATCGCTACGGGAATTTTCAAGCTAATGACGACGGCGTAAGAGCCGTCGCAGACGGTGGTTTATC
GTTGGGATCAAACGAGTGGGGGTATCAAGAAATGTTGATGTACGGAACTCAGTTAGGCTGTACTTGCCGAAGATCGTGGGGA
TAG
SEQ ID NO: 16: Physcomitrella patens PpWOX13LA
ATGACAAAGTCAGTTCCCCTGACTTCATTAATCCATGGTTATGCGATTCTCAGGACTGATCTCGATACCTTGGAGCCGTTGC
AAGGGATACATTGGAAATCAAGTCGATTGATCGAAAACAGGCAGAGCAACGGCATGGAATCTGAATCTAGGTTAGGTCGAAT
GATGGACATGACACCTTTGGGGTCGGGATTGCAAGGGCAACCTGTTCCTGGTGGAGCTGCGCTCGGCCTTGGGCCTTCGTTG
GAGAATTCGTTGCCGCAACCCATGTACACTCGGGGGTCTGGGCAGGTAATGACAGAAGAGCAGCTCGAAACATTGCGACGAC
AGATTTCGGTGTATGCAACAATCTGTCAACAACTTGTTGAAATGCACAAAGCGAGTGTTTCACAACAAGCATCTCTTCCTGG
CATTCTAGCAAGTGGTCAGATTGTGTCGATGGACCATCTCACTGGAACACCCCCTCACAAATCGACAGCAAGACAGCGGTGG
ACCCCCAGCCAACATCAGCTGCAAATTTTAGAAAAGTTGTTTGAGCAAGGCAGTGGCACACCCAACAAACAGCGCATTAAAG
AGATTACTGCCGAACTCAGTCAGCATGGTGCAATCTCGGAGACAAATGTGTACAACTGGTTTCAGAATCGCAAAGCCCGAGC
CAAAAGGAAGCAGCAATTGGTTACCCCAAGGGATGGTGAATCGGAAGCAGATACAGATGTAGAGTCACCAAAGGAAAAACGT
ACAAGACAGGAAGGTGAACAAAATCAGGACGAATCAGGGGGTGTTGGTGATACAAATGGTGGAGGCAACTCTGATGGAGCTG
GAAATGGGGTTCCTGAGCAAAGAGCTGCCAACTTTGACCAGCAGGATGCCGCTTCGTCTGCGCTGCTGCATTCACAAACAGA
TACTAAACCTGATATATCATCATTTAACAGGAGTGCTGGGTTCGATCCTCATAATGTATCTCAAGGCATCCCTCCCATGATG
AGTTAA
SEQ ID NO: 17: Arabidopsis thaliana AtESR1
ATGGAAAAAGCCTTGAGAAACTTCACCGAATCTACCCACTCACCAGACCCTAATCCTCTCACAAAATTCTTCACTGAACCTA
CAGCCTCACCTGTTAGCCGCAACCGCAAACTGTCTTCAAAAGATACCACTGTAACCATCGCCGGAGCTGGCAGCAGCACGAC
GAGGTACCGCGGCGTACGCCGGAGGCCGTGGGGACGATACGCGGCGGAGATACGTGACCCAATGTCGAAGGAGAGACGTTGG
CTCGGAACATTTGACACGGCGGAACAAGCCGCTTGTGCTTACGACTCTGCGGCTCGTGCCTTTCGTGGAGCAAAGGCTCGTA
CTAATTTTACTTATCCGACAGCTGTCATTATGCCTGAACCAAGGTTTTCTTTTTCCAACAAGAAATCTTCGCCGTCTGCTCG
TTGTCCTCTTCCTTCTCTACCGTTAGATTCCTCTACCCAAAACTTTTACGGTGCACCGGCAGCGCAGAGGATCTATAATACA
CAGTCTATCTTCTTACGCGACGCCTCGTGTTCCTCTCGTAAAACGACTCCGTATAATAACTCTTTCAACGGCTCATCATCTT
CTTACTCAGCATCGAAAACGGCATGCGTTTCTTATTCCGAAAACGAAAACAACGAGTCGTTTTTCCCGGAAGAATCTTCTGA
TACTGGTCTATTACAAGAGGTCGTTCAAGAGTTCTTGAAGAAAAATCGCGGCGTTCCTCCTTCTCCACCAACACCACCGCCG
GTGACTAGCCATCATGACAACTCTGGTTATTTCTCTAATCTCACTATATACTCTGAAAATATGGTTCAAGAGACTAAGGAGA
CTTTGTCGTCGAAACTAGATCGCTACGGGAATTTTCAAGCTAATGACGACGGCGTAAGAGCCGTCGCAGACGGTGGTTTATC
GTTGGGATCAAACGAGTGGGGGTATCAAGAAATGTTGATGTACGGAACTCAGTTAGGCTGTACTTGCCGAAGATCGTGGGGA
TAGCTAGATATTCATCATGATTATGTTTTGAGTTTTGGTACTATCGACTTAGTTTAAAGTTGCTACCTTTCCCAATGTTGGA
TATTAACTAAATTATGTTTTAAGTTGAATTTGCTAATAGGATTTCATAATTATAATCAAGTTTATAATATATTTTCGTAGCT
AATTAAAGTTTATATCCACGTATTCTGACACATTACGCGCTT

The terms “percent identity” or “identity” in the context of two or more nucleic acids or polypeptides refer to two or more sequences that are the same or have a specified percentage of nucleotides or amino acid residues that are the same. The percent identity can be measured using sequence comparison software or algorithms or by visual inspection.

In general, percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. With regard to DR sequences, the total number of aligned nucleotides or amino acids refers to the minimum number of DR nucleotides or amino acids that are necessary to align the second sequence, and does not include alignment (e.g., forced alignment) with non-DR sequences. The total number of aligned nucleotides or amino acids may correspond to the entire DR sequence or may correspond to fragments of a full-length DR sequence.

Sequences can be aligned using the algorithm described by Altschul et al. (Nucleic Acids Res, 25:3389-3402, 1997) as incorporated into BLAST (basic local alignment search tool) programs, available at ncbi.nlm.nih.gov on the World Wide Web. BLAST searches or alignments can be performed to determine percent sequence identity between a DR nucleic acid or amino acid sequence and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a query sequence and another sequence, the default parameters of the respective programs are used.

The plant transformation methods provided herein also can be used to deliver genome editing reagents to leguminous plants. Genome editing reagents include, without limitation, sequence-specific nucleases such as meganucleases, zinc finger nucleases (ZFNs), transcription activator-like effector (TALE) nucleases, and clustered regularly-interspaced short palindromic repeats (CRISPR)/CRISPR-associated (Cas) nuclease systems, and DNA base editors (e.g., a cytosine deaminase or adenosine deaminase such as BE3 or ABE). Materials and methods for using such genome editing reagents are found, for example, in U.S. Pat. No. 8,586,363, and U.S. Publication Nos. 2015/0167000, 2016/0237451, 2019/0249183, 2015/0166981, and 2015/0166980, and in Komor et al., Nature 533(7603):420-424, 2016. Upon delivery, a nucleic acid encoding a genome editing reagent can either integrate into the genome or can be transiently expressed in the plant cell without integration, can be expressed, and can then generate edits at the target sequences.

In some cases, a genome editing reagent can be a Cas9 endonuclease. The Cas9 protein includes two distinct active sites—a RuvC-like nuclease domain and a HNH-like nuclease domain, which generate site-specific nicks on opposite DNA strands (Gasiunus et al., Proc Natl Acad Sci USA 109(39):E2579-E2586, 2012). The RuvC-like domain is near the amino terminus of the Cas9 protein and is thought to cleave the target DNA that is noncomplementary to the crRNA, while the HNH-like domain is in the middle of the protein and is thought to cleave the target DNA that is complementary to the crRNA. A representative Cas9 sequence from Streptococcus thermophilus is set forth in SEQ ID NO:23 (see, also, UniProtKB number Q03JI6), and a representative Cas9 sequence from S. pyogenes is set forth in SEQ ID NO:24 (see, also, UniProtKB number Q99ZW2).

SEQ ID NO: 23 (S. thermophilus):
MTKPYSIGLDIGTNSVGWAVTTDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKAYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEV
FKDDTKNGYAGYIDGKTNQEDFYVYLKKLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSMDNNALQNDRLYLYYLQNGKDMYTGDDLDIDRLSNYDIDHIIPQ
AFLKDNSIDNKVLVSSASNRGKSDDVPSLEVVKKRKTFWYQLLKSKLISQ
RKFDNLTKAERGGLSPEDKAGFIQRQLVETRQITKHVARLLDEKFNNKKD
ENNRAVRTVKIITLKSTLVSQFRKDFELYKVREINDFHHAHDAYLNAVVA
SALLKKYPKLEPEFVYGDYPKYNSFRERKSATEKVYFYSNIMNIFKKSIS
LADGRVIERPLIEVNEETGESVWNKESDLATVRRVLSYPQVNVVKKVEEQ
NHGLDRGKPKGLFNANLSSKPKPNSNENLVGAKEYLDPKKYGGYAGISNS
FTVLVKGTIEKGAKKKITNVLEFQGISILDRINYRKDKLNFLLEKGYKDI
ELIIELPKYSLFELSDGSRRMLASILSTNNKRGEIHKGNQIFLSQKFVKL
LYHAKRISNTINENHRKYVENHKKEFEELFYYILEFNENYVGAKKNGKLL
NSAFQSWQNHSIDELCSSFIGPTGSERKGLFELTSRGSAADFEFLGVKIP
RYRDYTPSSLLKDATLIHQSVTGLYETRIDLAKLGEG
SEQ ID NO: 24 (S. pyogenes):
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFTERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

Thus, the materials and methods provided herein can utilize a Cas9 polypeptide having the sequence of SEQ ID NO:23 or SEQ ID NO:24. In some embodiments, however, the methods described herein can be carried out using a Cas9 functional variant. Thus, in some embodiments, a Cas9 polypeptide can contain one or more amino acid substitutions, deletions, or additions as compared to the sequence set forth in SEQ ID NO:23 or SEQ ID NO:24. In certain cases, polypeptides containing such changes can have at least 80% (e.g., at least 85%, 90%, 91%, 92%, 93%, 94%, 95%, 96%, 97%, 98%, or 99%) sequence identity to SEQ ID NO:23 or SEQ ID NO:24. The activity of a functional Cas9 variant may be altered as compared to the corresponding unmodified Cas9 polypeptide. For example, by modifying specific amino acids in the Cas9 protein that are responsible for DNA cleavage, the Cas9 can function as a DNA nickase (Jinek et al., Science 337:816-821, 2012).

In some embodiments, therefore, a Cas9 protein may not have double-stranded nuclease activity, but may have nickase activity such that it can generate one or more single strand nicks within a preselected target sequence when complexed with a gRNA. For example, a Cas9 polypeptide can have a D10A substitution in which an alanine residue is substituted for the aspartic acid at position 10 (underlined in SEQ ID NOS:23 and 24), resulting in a nickase. In some cases, a Cas9 polypeptide based on the S. pyogenes sequence can have an H840A substitution in which an alanine residue is substituted for the histidine at position 840 (underlined in SEQ ID NO:24), resulting in a “nuclease-dead” Cas9 that has neither nuclease nor nickase activity, but can bind to a preselected target sequence when complexed with a gRNA. A Cas9 polypeptide also can include a combination of D10A and H840A substitutions, or D10A, D839A, H840A, and N863A substitutions. See, e.g., Mali et al., Nature Biotechnol, 31:833-838, 2013.

In some cases, amino acid substitutions within DR or endonuclease/nickase polypeptides can be made by selecting conservative substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. In some embodiments, an amino acid substitution can be non-conservative, such that a member of one of the amino acid classes described above is exchanged for a member of another class.

Any appropriate method can be used to transform or infect plants, plant parts, or plant cells with R. rhizogenes, including those described in the Example herein. R. rhizogenes can infect a wide range of plants, including many different soybean varieties and other legume species. The methods and systems provided herein therefore can be used in, without limitation, plants such as beans, soybeans, peas, chickpea, cowpea, pigeon pea, peanut, ground nuts, lentil, green gram, and black gram. As such, the methods described herein can serve as genotype-independent genetic transformation and genome engineering approaches that enable genetic transformation and genome modification in different soybean varieties, including commercial elite lines, and also in other legume crops that are recalcitrant to current plant transformation and regeneration technologies.

The invention will be further described in the following example, which does not limit the scope of the invention described in the claims.

### Example

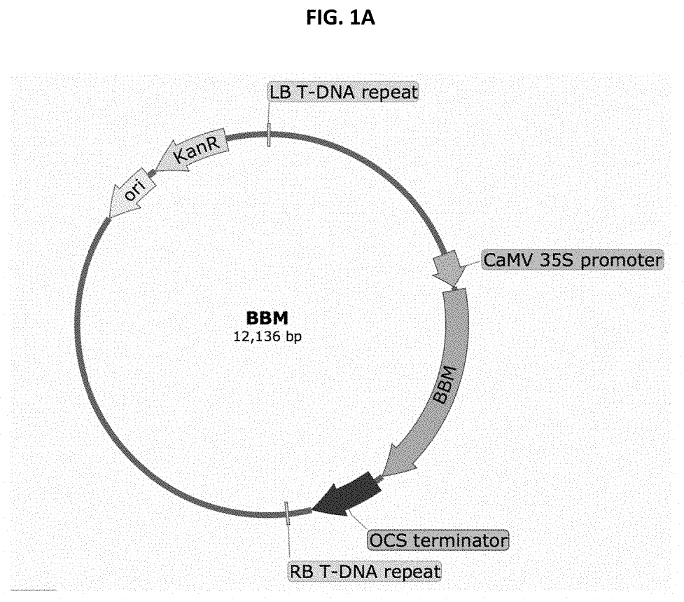

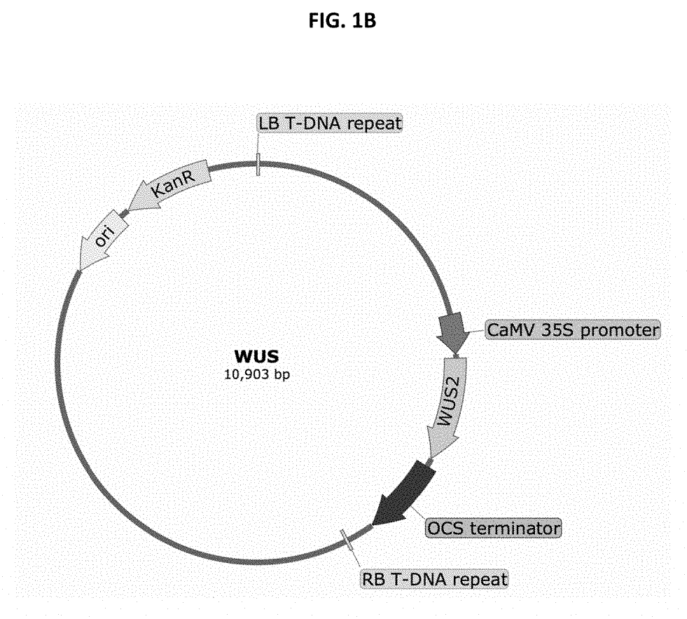

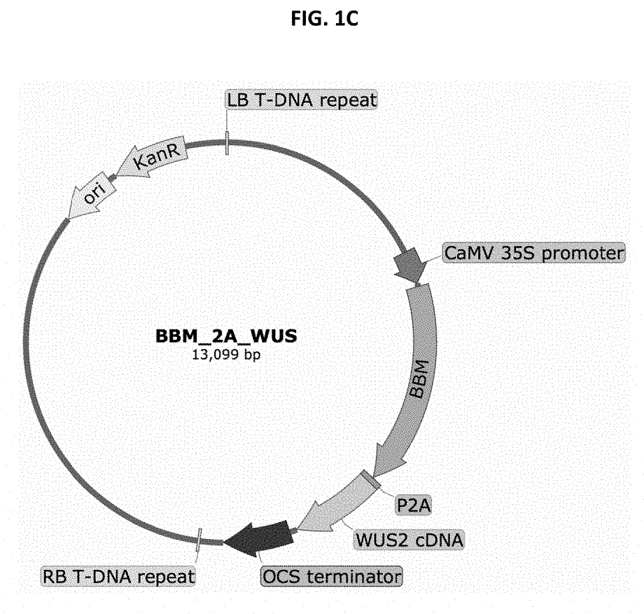

Sequences encoding the developmental regulators BBM and WUS, driven by the 35S promoter from cauliflower mosaic virus, were cloned into T-DNA construct either individually (FIGS. 1A and 1B, respectively) or together as a single expression unit separated by a 2A sequence (FIG. 1C). These T-DNA vectors were transformed into the 18r12 strain using a freeze and thaw method. A single colony from each transformation was inoculated in 50 mL LB liquid medium (with 50 μg/mL kanamycin) and incubated with rigorous shaking at 28° C. until the OD₆₀₀ reached 0.8-1.0. Cultures were pelleted by centrifugation at 4,000 rpm for 10 minutes, and then re-suspended in co-cultivation (CCM) medium with the volume adjusted to OD₆₀₀ of 0.6 for plant transformation.

To transform soybean (cv. Williams 82) cotyledons, dry soybean seeds were sterilized using vapor-phase sterilization (Liu et al., Methods Mol Biol, 1917:217-234, 2019), and placed on filter paper presoaked with ½×Murashige and Skoog (MS) liquid medium (pH 5.7) in Petri dishes in a culture room under 18/6 (light/dark cycle) photoperiod at 25° C. Typically, five- to seven-day-old cotyledons of germinated seeds were excised with a scalpel about 3 mm above the cotyledonary node. The adaxial side (i.e., the flat side) was cut gently multiple times at 1 to 3 mm depth to introduce multiple wounds. The wounded cotyledonary explants were submerged in Petri plates containing the rhizogenes culture. Cotyledons were incubated at room temperature for 20 minutes with occasional shaking. Inoculated cotyledons were placed adaxial side down on a single layer of filter paper presoaked with CCM liquid medium in Petri plates. The Petri dishes were wrapped with parafilm and incubated at 24° C. under an 18:6 (light:dark cycle) photoperiod for 5 days.

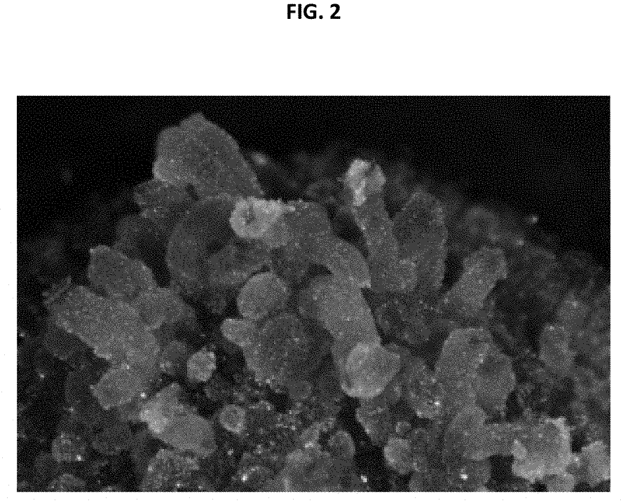

Four different 18r12 rhizogenes strains were developed and designated based on the DR or reporter genes in the transformed T-DNA constructs: BBM, WUS, BBM-2A-WUS, and Luc. The Luc strain contained a T-DNA harboring only a luciferase reporter gene, without any DR coding sequences. During transformation, soybean cotyledons were divided into 5 groups. As summarized in TABLE 1, four groups were transformed with single strains while one group, BBM/WUS, was transformed by mixing the BBM and WUS strains together in a 1:1 ratio. After about 10 days, shoot-meristem structures started to emerge on cotyledons infected by DR strains as shown in FIG. 2. The frequency of cotyledons that contained shoot meristem structures was scored and is summarized in TABLE 1. Compared to the no DR control Luc strain, the DR strains induced a high frequency of shoot meristem formation, ranging from 32% to 73%. Interestingly, the transformation groups with the WUS gene all exhibited remarkably high shoot meristem induction frequency (68% to 73%), while the group with only the BBM gene appeared to be less effective, with an induction frequency of 32%. Very few root formations were observed in either transformation group.

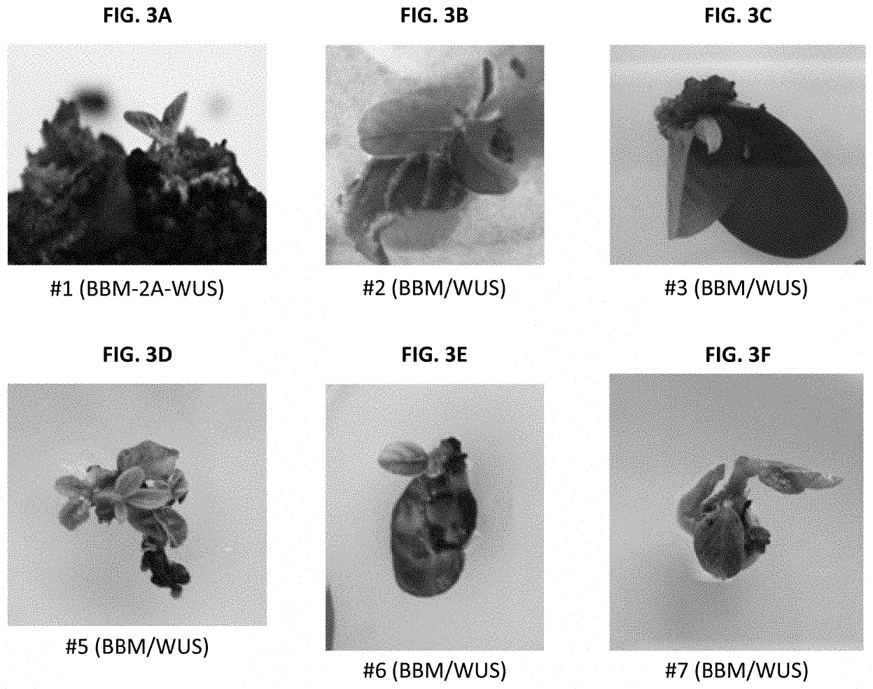

After 4 weeks of transformation, regenerated shoots with true leaves started to emerge from the three transformation groups with the highest frequency of shoot meristem structures (TABLE 1; examples shown in FIGS. 3A-3F). The transformation group with the single strain infection of WUS produced one regenerated shoot out of 52 cotyledons (1.9%). The transformation group with the single strain infection of BBM-2A-WUS showed similar results, producing one regenerated shoot out of 53 cotyledons (also 1.9%). In contrast, six shoots (5.9%) were regenerated from 97 cotyledons in the dual strain infected group, BBM/WUS, suggesting that the mixed strain infection could provide a more effective method for inducing shoot regeneration from plant tissues, such as cotyledons. Regenerated shoots were transferred into shoot elongation media until they reached 3 cm in length, and were then excised from the cell cluster/shoot pad and transferred to rooting media (RM) (Liu et al., supra) in a Combiness filter box. To obtain optimal root formation, the shoots and cell cluster pads were dipped into 1 mg/mL indole-3-butyric acid (IBA) for 30 to 60 seconds prior to culturing on rooting media. After a few primary and secondary roots formed from the bottom of the shoots (about 2-3 weeks), the plants were transferred into soil and grown to maturity.

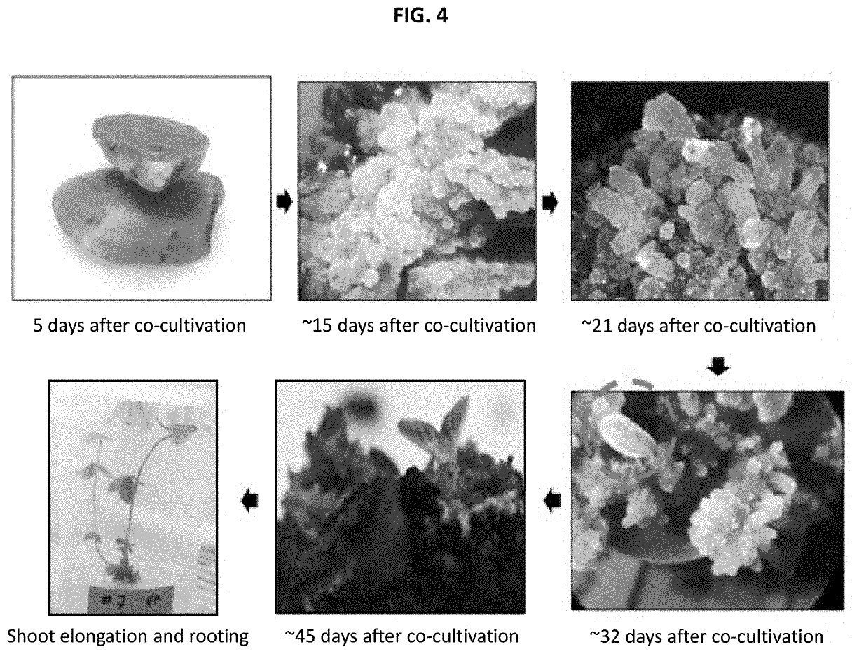

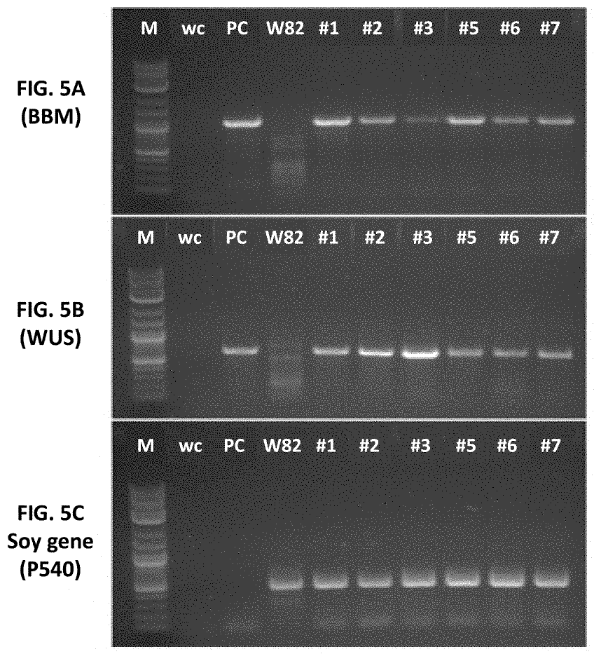

The steps and timeline of soybean shoot regeneration using DR-delivering rhizogenes bacterium are summarized in FIG. 4. Because this method is able to deliver genetic material into plants, it is possible to create transgenic plants using this process. To determine whether transgene sequences were integrated into the soybean genome, the regenerated shoots were characterized using a genomic polymerase chain reaction (PCR) assay. Leaf tissue was excised from six regenerated shoots, five from the BBM/Wus group and one from the BBM-2A-Wus group. Genomic DNA was isolated from each leaf sample. PCR analysis was performed using gene specific primers to amplify the BBM and Wus sequences (FIGS. 5A and 5B, respectively), along with negative (no DNA template) and positive (a cytochrome P450 gene endogenous to the soybean genome; FIG. 5C) PCR controls. All regenerated plants appeared to contain transgene sequences (FIGS. 5A and 5B). Further characterization of the progeny from these plants is conducted to confirm whether the T-DNA transgenes are heritable to the next generation.

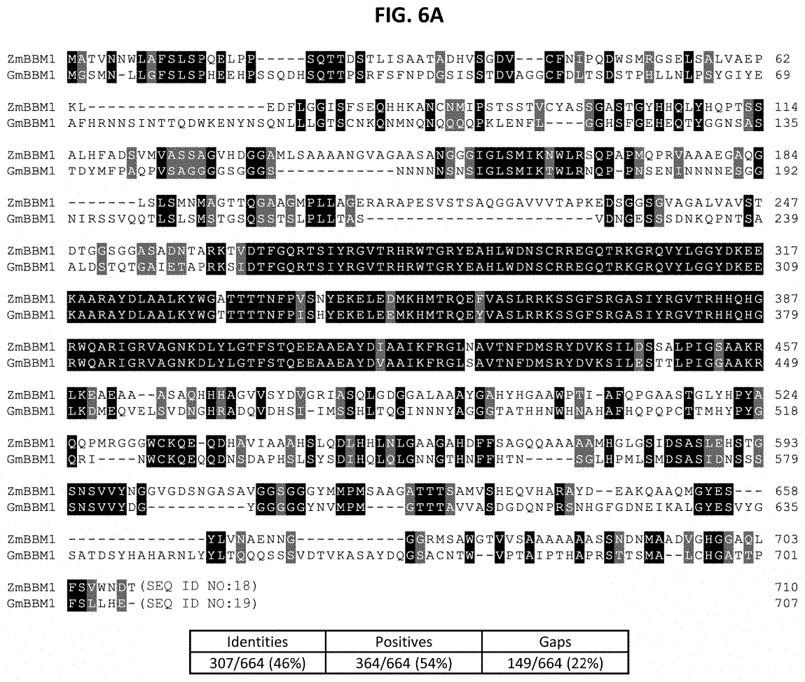

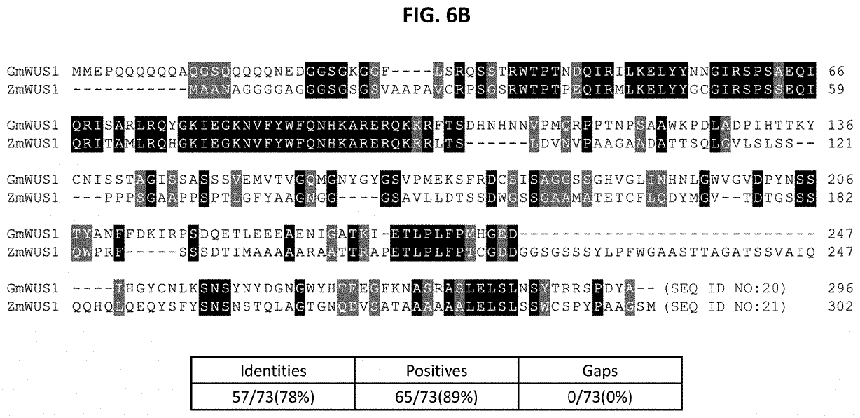

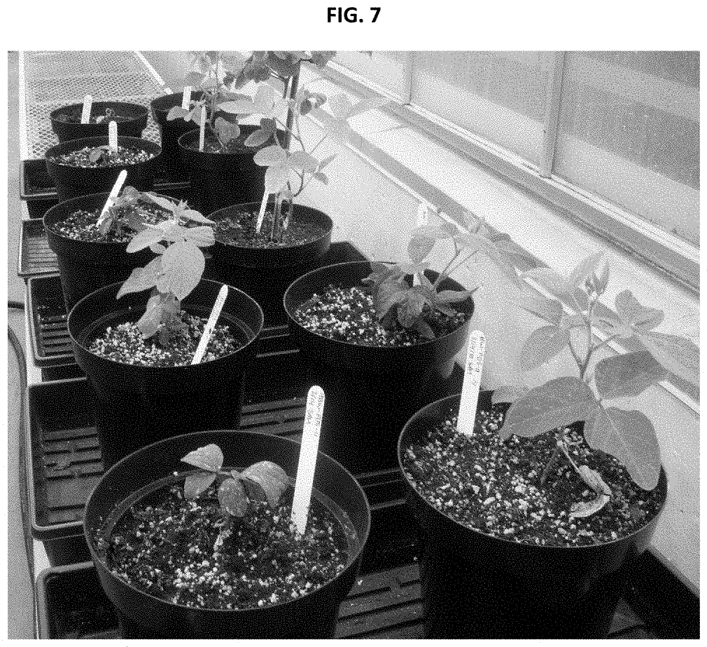

The BBM and Wus developmental regulator homologous genes from maize (zmBBM and zmWus) and soybean (gmBBM and gmWus) were compared for the efficacy of promoting soybean shoot regeneration. Amino acid sequence alignments between these homologous genes (FIGS. 6A and 6B) showed that the sequence identities are 46% between the BBM homologous genes and 78% between the Wus genes, respectively. The soybean BBM and Wus homologous genes induced shoot regeneration with an efficiency of 10.1%, which was 1.7-fold higher than the efficiency for the maize BBM and Wus genes (TABLE 1). Regenerated shoots induced by the developmental regulator genes from both species were grown in soil to set seeds, and no abnormal phenotypes were observed (FIG. 7). Two soybean cultivars, W82 and Maverick (TABLE 1), were tested in these studies. Notably, the BBM and Wus genes induced efficient shoot regeneration in the different cultivars, indicating genotype independency of this methodology.

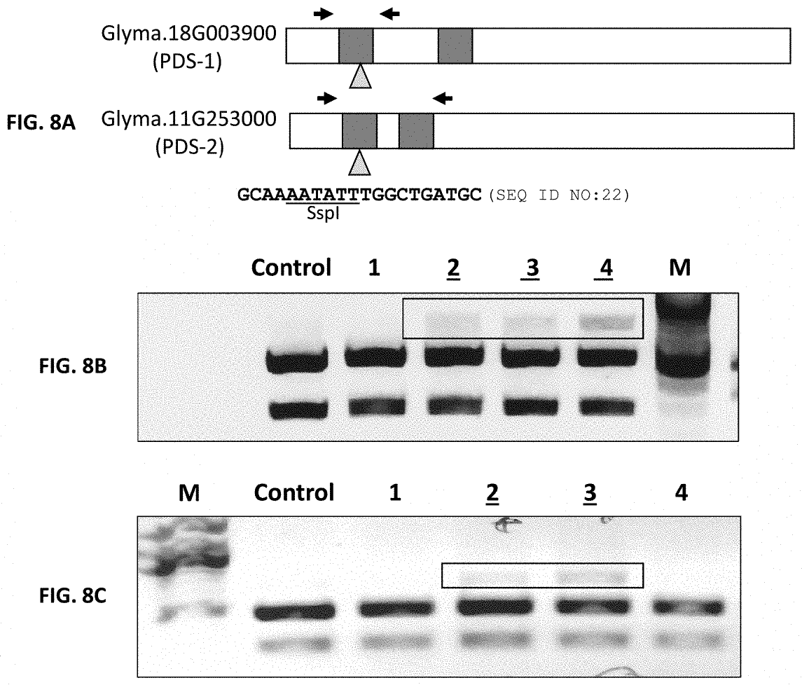

Targeted gene modification (e.g., gene editing) was tested using R. rhizogenes strain K599. K599 contains root-inducing genes that promote root formation in a phenomenon also known as hairy roots. Two copies of the phytoene desaturase (PDS) genes were identified in the soybean genome. Plants with homozygous disruption of both gene copies display an albino phenotype that can serve as a visual marker to quickly assess gene editing efficiency. A CRISPR guide RNA was designed to target an identical region between these two PDS genes (SEQ ID NO:22; FIG. 8A), and was cloned into a T-DNA construct containing a Cas9 endonuclease cassette encoding a S. pyogenes Cas9 enzyme. The resulting CRISPR/Cas9 expressing T-DNA was transformed into the K599 strain and then transfected into soybean cotyledons using the method described above. The regenerated roots that were formed within 2 weeks were collected for analysis. Genomic DNA was isolated from 4 individual root samples, and PCR was performed using PDS gene specific primers to amplify the CRISPR targeted regions from each PDS locus. The PCR amplicons were then digested by the restriction enzyme, SspI, as both PDS loci had an SspI recognition site overlapped with the CRISPR targeted sites, and targeted mutations introduced by CRISPR/Cas9 in these regions disrupted the restriction site. The presence of PCR amplicons that were resistant to restriction enzyme digestion indicated the occurrence of targeted gene modifications. These studies showed that 3 out of the 4 root samples showed mutations in the PDS1 gene, and 2 out of the 4 root samples displayed mutations in the PDS2 gene (FIGS. 8B and 8C). Together, these data indicated that the CRISPR/Cas9 system was able to induce targeted mutations in the regenerated tissues using the R. rhizogenes mediated transformation approach. It is anticipated that these mutations will be present in regenerated shoots when constructs containing developmental regulator sequences as described herein are co-transformed with the targeted nucleases.

TABLE 1
Summary of shoot regeneration efficiency from each transformation group

| | | | | Frequency of | | Frequency of |
|---|---|---|---|---|---|---|
| | | | No. of | shoot meristem | No. of | shoot |
| Cultivar | Transformation group | Construct | Cotyledons | formation | shoots | regeneration |
| W82 | Luc | 35S::ZmLuc | 55 | 7 | 0 | 0.0 |
| W82 | ZmBBM-2A-ZmWus | 35S::ZmBBM-2A-ZmWUS | 53 | 68 | 1 | 1.9 |
| W82 | ZmBBM | 35S::ZmBBM | 53 | 32 | 0 | 0.0 |
| W82 | ZmWus | 35S::ZmWUS | 52 | 69 | 1 | 1.9 |
| W82 | ZmBBM/ZmWus | (35S::ZmBBM) + (35S::ZmWUS) | 97 | 73 | 6 | 6.1 |
| Maverick | ZmBBM/ZmWus | (35S::ZmBBM) + (35S::ZmWUS) | 64 | 69 | 4 | 6.0 |
| Maverick | GmBBM/GmWUS | (35S::GmBBM) + (35S::GmWUS) | 158 | 75 | 16 | 10.1% |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for generating plant tissue comprising one or more genetic modifications of interest, the method comprising:
(a) using a Rhizobium rhizogenes strain to introduce, into non-meristematic tissue of a leguminous plant, (i) nucleic acid encoding one or more developmental regulators and (ii) nucleic acid comprising one or more sequences that, when expressed, modify cells within the plant to achieve the one or more genetic modifications of interest, wherein expression of the one or more developmental regulators induces shoot formation from the non-meristematic tissue; and
(b) culturing the shoot induced by the one or more developmental regulators, to obtain modified plant tissue comprising the one or more genetic modifications of interest.

2. The method of claim 1, wherein the R. rhizogenes strain is selected from the group consisting of 18r12, K599, A4, R1000, R1200, R13333, R15834, R1601 and LBA9402.

3. The method of claim 1, wherein the R. rhizogenes strain is 18r12.

4. The method of claim 1, wherein the one or more developmental regulators comprise one or more of isopentenyl transferase (IPT), Baby Boom (BBM), Shoot Meristemless (STM), Leafy Cotyledon (LEC), Wuschel (WUS), WUS homeobox-containing (Wox), and APETALA2/Ethylene Responsive Factor (AP2/ERF) factors.

5. The method of claim 1, wherein the non-meristematic tissue comprises a cotyledon or a portion thereof.

6. The method of claim 1, comprising introducing nucleic acid encoding two or more developmental regulators, wherein the two or more developmental regulators are encoded by one plasmid or one T-DNA.

7. The method of claim 6, wherein the two or more developmental regulators comprise BBM and WUS, WUS and IPT, WUS and STM, WUS and LEC, or WUS and ESR1 and WIND1.

8. The method of claim 1, comprising introducing nucleic acid encoding two or more developmental regulators, wherein the two or more developmental regulators are encoded by separate plasmids or separate T-DNAs.

9. The method of claim 8, wherein the two or more developmental regulators comprise BBM and WUS, WUS and IPT, WUS and STM, WUS and LEC, or WUS and ESR1 and WIND1.

10. The method of claim 1, wherein the leguminous plant is selected from the group consisting of common beans, soybeans, peas, chickpea, cowpea, pigeon pea, peanut, ground nuts, lentil, green gram, and black gram.

11. The method of claim 1, wherein the one or more genetic modifications comprise insertion of a transgene that, when expressed, edits the plant cell DNA.

12. The method of claim 11, wherein the nucleic acid that modifies a plant cell encodes a targeted endonuclease.

13. The method of claim 12, wherein the targeted endonuclease comprises a meganuclease, zinc finger nuclease, transcription activator-like effector nuclease, or Clustered Regularly-Interspaced Short Palindromic Repeats-associated nuclease with a guide RNA.

14. The method of claim 1, wherein the one or more genetic modifications comprise insertion of a transgene that, when expressed, confers an agronomic trait.

15. The method of claim 1, wherein the nucleic acid that modifies a plant cell encodes a targeted enzyme that modifies plant DNA.

16. The method of claim 15, wherein the nucleic acid that modifies a plant cell encodes a targeted endonuclease.

17. The method of claim 16, wherein the targeted endonuclease comprises a meganuclease, zinc finger nuclease, transcription activator-like effector nuclease, or Clustered Regularly-Interspaced Short Palindromic Repeats-associated nuclease with a guide RNA.

18. The method of claim 1, further comprising assaying shoot tissue induced by the one or more developmental regulators for the one or more genetic modifications of interest.

19. The method of claim 1, comprising placing the shoot induced by the one or more developmental regulators into culture and inducing the shoot in culture to form a plant.


SEQUENCE LISTING (24 sequences)

SEQ ID NO: 1 - DNA, 842 bp, Cauliflower mosaic virus
agatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggact

SEQ ID NO: 2 - DNA, 304 bp, Agrobacterium tumefaciens
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccgta

SEQ ID NO: 3 - DNA, 2154 bp, Glycine max
aaaattatttatttatatgttaataaaattatttaacttttaagaaaactaatacaagacgaagaaaaattcttccaccacatttttctctatctcattttttaaataaatattaaaaatatagtattttcttcctatatttcctctgatttcattcttatttatgtatgttaaattcttattttttcattcatccgtataaatcaaactcacatatgaataggttttaatatttcacgcacacacactgttgaaccaaccgagctagaccttttcattactcttattttttttgttgaattaattaaaaaaaaaaaagcaaaactgatagaaagtttcataaatcaagtacaaaacagattatgttattcatctacacaatacaatcttgaaagaagtttgcgggaaagatgagactggtgacaattcatgactgaagcatgctggttgtttactgattacaatcaattagtatatggggttgtaatatttattgtattgtaccgaatttcagcacatcatgtgttgggttttgcatctcctaattcaataaaattagtttgccttcaataaagcaaaagtgatggagtctgcgagctaaaagtttgcatctaagacaaatttgtaagtttaagtattattatattatagatgagttaaagtagtatttgatgtaattattttaatttttcttactttgaaaagaagtttgattaaatacattatcttgaaaacactaaaaaaaattacagattaaaaataattttaaggagaaactagatgaagtgaagttagctagaggataaaaactttttccctcactctgttccccacactgatctttcttttgtctctgcactttctcttttttttttctatctccttactctctctctctctctccccactttttactgagtttctttttaaagttaatagagaaaataaattttataaaggataacgatactcctggtctgcagttcatttttgggtagataaaatggatgcaagagaattatagaaagacaagcaaggaagaaaattttatttatgggcagataaaattgatggaagagaatgatagaaaggaagaaaaaaaatatcgactatggaaggatggttttaacaacactttttttctaacattttttttattgactaaaatttattataaattacacatgttttaaaatcttacttttaaatcaagagggacatatagtcattaagcaagaatgagacctattaaaatttataatttttaataaattttaactaataataaaatagagagtgtatattaaaaagaaagtagaggttacaaagaataattttactaccatttctctcaaagtctcaacgtcactaacggatttacatggaaattaatggggtaattgttcttacaaactaatttttttacttaaatatattaaataaatattttgttgtcatcataaaaaaaatagattttgtcttcataagataattttttaataagatgaatgtaaatagttgtaaaagataaaagagaaaataaattaatattaattttatcactttattcttttaaatcttactgtttttaatttttttttattaccatttttaataaatcttatatatttttttgtttgaaaaatattggactatttttgtcctactaaaaaatttctcaatccgccggtgctcaaaatgaaatatgttaccgacttcttgttcattagaatgtagagtctggaagatagataaaccaggggctgcatggaaaatgggtacagtatatatatatatatatgtaaattaatgctgcttcaattaattacttactataccttccttttgaaatctagacactagacatggttatggttacccttcacctcttgcctcagagtagagtaattgttgcatttaactaacccaaccacaatccattattacactgaattcatctcaactacccaataataagcaccaccacatgccccttctgcaattgcactgcaccccatagacacatagccccccttctcccatatataaacccaaacccaccacagtgtctcaaaacacaccacaccttcaaaccctaaccctagaagaagaaaaaaaccacaaccaccaccatctagggtttttctttttctc

SEQ ID NO: 4 - DNA, 2121 bp, Glycine max
gattttgtgtgtcaaagaaagtatgctcatgaaattctaggaaggtttgaaatggataaaagcaatccagtgaagaatcctattgttccaggcaggaaattgtcaaaggatgaagcaggaaccaaagttgatgaaactttgttcaaacaggtagttggcagtctgatgtacttgactgcaactcaacctgatttgatgtatggagtgagtcttattagtagattcatgtcttgcccaactgaatctcattggcttgcagcaaaaagaatattaaggtatttgaaaggtacaactgagcttggaattttctacaaaaaggatggttgcacaaatttggtggcttacaccaacagtgattttgttggtgatttggatgatcgtaggagcacttctggctttgcgttcttacttggttctggagcagtttcatggtcttctaaaaaacaacctatagtaacattgtctactactgaggctgagtacatagctgctgcgttttgtgtctgtcagtgtatttggttgagaagagttttagagaaacttggtcataaggaggaaaatagtactttgattcaatgtgacaataattctactatacaactatcaaagaattttgtttttcatggcagaagcaaacatattaatacaaggtttcattttcttagagatttgaccagagacaaaattgtggagttgagttactgtaattctcaagaacaggttgcagacataatgacaaaacctctcaagttagaacaattcttgaaattacggagtatgttgggaatggttgatgtgtcagttataaactaaatgtttgtttttctttttagtttaaggaagggaatgttgacgtgtaaatagaagttaatagaagttaagaagttagcggtagttttttgtaaataggagttatgtggagtagttatatggagtagttatgtggagtagttataaatatattgtaactgcagtggtctatatattgtacctttgtaactatagtaattgaatttgaagattaaaaaaaagagcattctctctaaaacttgtatgtcattgttctttttaacaataatatactagtacgtacttaaacttacaatctctaaaaaaaaatacttaaacttacaatttgttttagaagcaaacttaactcacaagctccatgccttcatgatgaaattattttaatttttctttctttgaaaagaggttggattaaatacgttatcttgaaaactctaaaaaaaattaatcagaataatctttatatataaaagaaaaaaaataattaattttaaaaagaaactagatgaagtgaagttagaggatagatttttttccctcactctgttcccactctttcttttgtctctacactttctctttttttatgtctcctaactcgatctccattttttactgagtttcttttttaagttaaaagagaaaataaatatttttatagaatctctatccatttcatctactcctgcacttcagttttgggcagatgaaatggatgaaaagagagttatagaaagaaaagaaaaggaagaaaaattatttttgggaagataaaatggatggaaggttgttgttcatcagaatctggagtctggaagatagataaacccggggctgcatggaaaatgagtaaagtgatttacttcttgacacgtaacactgatatatatatatatatatatatatatatatatatatatatatatatatatatatattccagttcagtacacataaatatcaatgttgcttcaatgaattactacaacttcctttgaaatctagacacatggttatggttacccttcacttcttgcctcagagtatagtaattatagcatttaacttacccaaccacaatccattattacactgaattcatctcaactacccaaataagcaccacccacatgccccttctgcctgcactgcaccccatagacacatagtcccatagccttctcccaatatataagcccaaccctaaacccaccacatagtagtttctcaaaacacatcacaccttcaaacccgaaccctaaccctagaagaaagaaaaaaacccacaacacacaatcagtactaccaact

SEQ ID NO: 5 - DNA, 2188 bp, Glycine max
tggagacaacacacagaaacaccagacagtaaggttttcataggcaccgaaaagcgtgaaaagtgtgcaaaatcataagctagcctgttcacaattgacttgtgcgtcactagttttgctaaacactgaccagctcataccaatttgaatacatttgtgatcaaataagtggctcaaacatgataggtgaccgattttcattcaaaccgattgaatcggtcgatgaaaatggagcatgatcaataacacttggcgaaaggagtaagaggccttacgagtgaagtggtgaaagagaatctttcttagagcaccaggatgagaaaggatggtataaatgttaacacgttacaatttgtttacgacacaattttctgttgcctaaccttgagtgggtaatgtcatcctccccaaagtaaatcagattccttttttttttttctaaaataaagatattttgttcaccacacataattttacgtttttgataaattgtagagatattatgttcatccagaaactatacaataacttcattattcttctcttttgttatcatatcacttgttattttatattttttctttttttgttttctattttttctatctttcttatttataaaaaaataatacacataaaataaaatttacatcatttttgcatctatttctctctatatcatttattatatattttttctttcctctccattggttgtttttttcaaaaaccaaaacttctttctctgcttgctagtcttaaaaattattgtataatttaggataaatagtcatttttgtctttaaatgtgtaattcgctcacaaatgtgtccctgaaagataaaaatataaaatttagtccttgaaaatgtaaaacatgcaacaagtatatccgatcattaacttccgtctggtaccgttaataaaataacttatatgacacatagggacgaattgtcactaacatgaatgattgtcaatgtggtcatctctatttgtcagcatagggacatatttgtcatataatattttttttttactttttgtcttctcattttgctgacaaatgtgtccctaagagataaaaatacaaaatttagttcccgaaagtgaaaaaaagatgacaaatatatccggacgttaatagtagattgtgattaattattataatttaaaaaaatttataatgattgcaacaaaattctgggagagctaaatcatattggtaagtttttgtttccattataaaaattttaagcgaagtaaaaaaaattactataaaacaataaaaaaatcatgatttttgtttaatcaaacactatttatttaattatttttttatagatttttcataataaaatatgaatctctattagtatatgcaatcacatcaaattataaattataataaaagtttttttattaactaatcatttaaaaaatcataactttaaaaaaataattccaaaggaggtgagtatttttgtacaaattaaaagtgtcaatgtaattgcaatgtttcctaattcgatctttttatcgtcatcagtataaaaaattttaaattataataattattcacaatctattattaatatctagatatatttatcagatttttttcacttttaatgaataaattttgtatattaatattttaagaatgtatttatcaatgacttttacaagtaactatttattactattatataagtatatttatctaaaaaaatgtataaaaacattacatatcaatgaagaaactagttattgcaggaccaattaataaaagaaatacaattcaatcctcgaaatctgtcaaggtatggagggataagctataatattaaatatatcatcctattcagtcgtatttggggcaccaccaaatcaagcgtcacatattaagccaggtgccaaaagattatactatgcatgcaccaccacctagcattaaattgagcacaactgccaccaaacaaaaaacagtactgccacatgcattttccataacccttagttaccttcccacagccctaggagtcctatcttatcttctatataactccccacacttgcaccacttcactcaccaaacaataagcaagaatttccattgcatgcaaagcaaatacgcattatattactagttttaatttgttttgtcgtttgcttctttctttgatta

SEQ ID NO: 6 - DNA, 1046 bp, Arabidopsis thaliana
cgcaaacgcagccattacaacgctatttcaaaacttatttaaacaattagtaatacttaccaaagcttttgaaagatactccagtaaacttgtcaaattcttatattgttctaatagactttataataaaacgtaagcatcgggcgatatggcatgattatttcatgacagaagaaataaataattttaacaaaaaaaaagacagaagcaataaataaaaaacaagcttctgatacatgaaatacatatatgtctcatacatacgtttagacaaacctgaaatgtcctcttcgtacaataatatccacaagtgtcagatttacactgaagtgttgctatgcatatcttttgttccatacttttcttacaaaatcatttattttcttctcgatatcaccgattggtgtatgttgtaatcatacattaacattaacaaacaaagatactttttcaaggattattctaattggcagttttagaaataatggaattatcctttactaatcatacagaaataatgtaaatatgtttagcatgtgtgaaatgactgttggtcgatttttaactttaataaataaaaaagcaataagaacgtggttttatttcgccactcccacttgcatcgtcatcatcaaagaaaaacactaatgtctagaccaaagatttaaaacatctaccccatatatatatgatgaacaagatagcaagtaaatttaaatgtaaaattaaattttagtttgctaagattaatatacaaaagaagtattatcaatttatcagttattaatcaaatcaagttttaagtgcaactcaaaagtttccatgcttatatagttatttgtatactactatactgtatgtgcaagaaaagcatttatactcttcgccatatatttcaaacttcacaaaatttaattaaatttttataccatttatgtacctataaatagatagaagaagctccatctctttcaaactatcaaccaccaaaatctttcacattacaccttccttttgtcctcaaaccaaaaccctagaaaccaaaa

SEQ ID NO: 7 - DNA, 909 bp, Zea maize
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctgtactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtgaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgcgatggccaccgagacatgcttcctccaggactacatgggcgtgacggacacgggcagctcgtcgcagtggccacgcttctcgtcgtcggacacgataatggcggcggccgcggcgcgggcggcgacgacgcgggcgcccgagactctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 8 - DNA, 1149 bp, Arabidopsis thaliana
atggagagtggttccaacagcacttcttgtccaatggcttttgccggggataatagtgatggtccgatgtgtcctatgatgatgatgatgccgcccatcatgacatcacatcaacatcatggtcatgatcatcaacatcaacaacaagaacatgatggttatgcatatcagtcacaccaccaacaaagtagttccctttttcttcaatcactagctcctccccaaggaactaagaacaaagttgcttcttcttcttctccttcctcttgtgctcctgcctattctctaatggagatccatcataacgaaatcgttgcaggaggaatcaacccttgctcctcttcctcttcttcagcctctgtcaaggccaagatcatggctcatcctcactaccaccgcctcttggccgcttatgtcaattgtcagaaggttggagcaccaccggaggttgtggcgaggctagaggaggcatgctcgtctgccgcagccgctgccgcatctatgggaccaacaggatgtctaggtgaagatccagggcttgatcaattcatggaagcttactgtgaaatgctcgttaagtatgagcaagagctctccaaacctttcaaggaagctatggtcttccttcaacgtgtcgagtgtcaattcaaatccctctctctatcctcaccttcctctttctccggttatggagagacagcaattgataggaacaataatgggtcatccgaggaagaagtcgatatgaacaatgaatttgtagatccacaagctgaggatagagagcttaaaggacagctcttgcgcaagtacagtggttacttagggagcctcaagcaagagttcatgaagaagaggaagaaaggaaagctccctaaagaagctcgtcaacaactgcttgattggtggagccgtcactacaaatggccttacccttcggagcaacaaaagctcgcccttgcggaatcaacggggctggaccagaaacagataaacaattggttcataaaccagaggaaacggcattggaagccgtcggaggacatgcagtttgtagtaatggacgcaacacatcctcaccattacttcatggataatgtcttgggcaatcctttcccaatggatcacatctcctccaccatgctttga

SEQ ID NO: 9 - DNA, 2133 bp, Zea maize
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgccagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatccacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctggaggacatgaagcacatgacaaggcaggagtttgtagcgcctctgagaaggaagtccagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagtccatcctggacagcagcgccctccccatcggcagcgccgccaagcgcctcaaggaggccgaggccgcagcgtccgcgcagcaccaccatgcgggtgtcgtttcctatgacgttgggaggattgccagccaactgggagatggcggtgccctcgctgcggcctatggtgctcactatcacggtgccgcgtggccaacgattgcattccagccgggcgcggcgtccaccggactgtaccatccttacgcgcagcagcctatgcgcggcggtggatggtgtaaacaagagcaagatcacgctgtgatagcagcggcacactccttgcaggatcttcatcatttgaatctcggagccgccggggcccacgactttttctcggcagggcagcaggccgccgccgctgcgatgcacggcctgggtagcatcgacagtgcgtcgctggagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtccatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtgggccacggcggcgcgcagctgttcagtgtctggaacgacacttaa

SEQ ID NO: 10 - DNA, 1007 bp, Arabidopsis thaliana
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaataacattaacaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggaaccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaa

SEQ ID NO: 11 - DNA, 723 bp, Agrobacterium tumefaciens
atggatctgcgtctaattttcggtccaacttgcacaggaaagacgtcgaccgcgatacgtcttgcccagcagactggccttccagtcctttcgctcgatcgggtccaatgctgtcctcaactgtcaaccggaagcggacgaccaacagtggaagaactgaaaggaacgacccgtctataccttgaagatcggcctctggtgaagggtatcatcgcagccaagcaagctcacgaaaggctgatcggggaagtgtacaattatgaggcccacggcgggcttattcttgagggaggatctatctcgttgctcaggtgcatggcgcaaagcagttattggagtaccgattttcgttggcatattattcgccacaagttagcagacgaggagacattcatgaacgcggccaaggccagagttaggcagatgttgcgccctgctgtaggcccatctattattcaagagttggttcatctttggaatgagcctcggctgaggcccatactgaaagagatcgacggatatcgatatgccatgttatttgctagccagaaccagatcacacccgatatgctattgcagcttgacccagatatggagggtgagttgattcatggaatcgctcaggagtatctcatccatgcgcgccggcaggagcaggaattccctccagtgagcgtggtcgctttcgaaggattcgaaggtccaccgttcggaatgtgctag

SEQ ID NO: 12 - DNA, 672 bp, Glycine max
atggaaactggaggctttcatggctaccgcaagctccccaacacaacctctgggttgaagctgtcagtgtcagacatgaacatgaacatgaggcagcagcaggtagcatcatcagatcagaactgcagcaaccacagtgcagcaggagaggagaacgaatgcacggtgagggagcaagacaggttcatgccaatcgctaacgtgatacggatcatgcgcaagattctccctccacacgcaaaaatctccgatgatgcaaaggagacaatccaagagtgcgtgtcggagtacatcagcttcatcaccggggaggccaacgagcgttgccagagggagcagcgcaagaccataaccgcagaggacgtgctttgggcaatgagtaagcttggattcgacgactacatcgaaccgttaaccatgtaccttcaccgctaccgtgagctggagggtgaccgcacctctatgaggggtgaaccgctcgggaagaggactgtggaatatgccacgcttgctactgcttttgtgccgccaccctttcatcaccacaatggctactttggtgctgccatgcccatggggacttacgttagggaaacgccaccaaatgctgcgtcatctcatcaccatcatggaatctccaatgctcatgaaccaaatgctcgctccatataa

SEQ ID NO: 13 - DNA, 891 bp, Glycine max
atgatggaacctcaacaacaacaacaacaagcacaagggagccaacaacaacaacaaaacgaggatggtggcagtggaaaaggggggtttctgagcaggcaaagtagtacacggtggactccaacaaacgaccagataagaatattgaaggaactttactacaacaatggaattagatccccgagtgcagagcagattcagaggatctctgctaggctgaggcagtacggtaagattgaaggcaagaatgtcttttattggttccagaaccacaaagctcgagaaaggcagaagaaaaggttcacttctgatcataatcataataatgtccccatgcaaagacccccaactaatccttctgctgcttggaaacctgatctagctgatcccattcacaccaccaagtattgtaacatctcttctactgcagggatctcttcggcatcatcttctgttgagatggttactgtgggacagatggggaattatgggtatggttctgtgcccatggagaaaagttttagggactgctcgatatcagctgggggtagcagtggccatgttggattaataaaccacaacttggggtgggttggtgtggacccatataattcctcaacctatgccaacttctttgacaaaataaggccaagtgatcaagaaacccttgaagaagaagcagagaacattggtgctactaagattgaaaccctccctttattccctatgcacggtgaggacatccatggctattgcaacctcaagtctaattcgtataactatgatggaaacggctggtatcatactgaagaagggttcaagaatgcttctcgtgcttccttggagctcagtctcaactcctacactcgcaggtctccagattatgcttaa

SEQ ID NO: 14 - DNA, 2124 bp, Glycine max
atggggtctatgaatttgttaggtttttctctctctcctcacgaagaacacccttctagtcaagatcactctcaaacgacaccttctcgttttagcttcaaccctgatggatcaatctcaagcactgatgtagcaggaggctgctttgatctcacttctgactcaactcctcatttacttaaccttccttcttatggcatatacgaagcatttcacagaaacaatagtattaacaccactcaagattggaaggagaactacaacagccaaaatttgctattgggaacttcgtgcaataaacaaaacatgaaccaaaaccaacagcaacagccaaagcttgaaaacttcctcggtggacactcatttggcgaacatgagcaaacctacggtggtaactcagcctctacagattacatgtttcctgctcagccagtatcggctggtggtggtggtagtggtggtggcagtaacaataacaacaacagtaactccatagggttatccatgataaagacatggttgaggaaccaaccaccgaactcagaaaacatcaacaacaacaatgaaagtggtggcaatattagaagcagtgtgcagcaaactctatcactttccatgagtactggttcacaatcaagcacatcactgccccttctcactgctagtgtggataatggagagagttcttctgataacaaacaaccaaacacctcggctgcacttgattccacccaaaccggagccattgaaactgcacccagaaagtccattgacacttttggacagagaacttctatctaccgtggtgtaacaaggcataggtggacggggaggtacgaggctcacctgtgggataatagttgtagaagagagggacagactcgcaaaggaaggcaagtttacttgggtggttatgataaagaagaaaaggcagctagagcctacgatttggcagcactaaaatactggggaacaaccacaacaacaaattttccaattagccactatgagaaagagttggaagaaatgaagcacatgactaggcaagagtacgttgcgtcattgagaaggaagagtagtgggttttctcgcggtgcatccatttatcgaggagtgacgagacaccaccaacatggaaggtggcaagcgaggattggaagagttgctggcaacaaggatctttacttgggaacttttagcacccaagaagaggcagcggaagcatatgatgtagcagcaatcaaattccgaggactaagtgctgttacaaactttgacatgagcagatatgacgtgaaaagcatacttgagagcaccactttgccaataggtggtgctgcaaagcgtttgaaggatatggagcaggttgaactgagtgtggataatggtcatagagcagatcaagtagatcatagtatcatcatgagttctcacctaactcaaggaatcaataacaactatgcaggagggggaacagcaactcatcataactggcacaatgctcatgcattccaccaacctcaaccttgcaccaccatgcactacccttatggacaaagaattaattggtgcaagcaagaacaacaagacaactctgatgcccctcactctttgtcttattcagatattcatcaacttcagctagggaacaatggaacacataacttctttcacacaaattcagggttgcaccctatgttgagcatggattctgcttccattgacaatagctcttcttctaactcggttgtttatgatggttatggaggtggtgggggctacaatgtgatgcctatgggaactactactgctgttgttgcaagtgatggtgatcaaaatccaagaagcaatcatggttttggtgataatgagataaaagcacttggttatgaaagtgtgtatggctctgcaactgattcttatcatgcacatgcaaggaacttgtattatcttactcaacagcaatcatcttctgttgatacagtgaaggctagtgcatatgatcaagggtctgcatgcaatacttgggttccaactgctattccaactcatgcacccagatcaactactagtatggctctctgccatggggctactacacccttctctttattgcatgaatag

SEQ ID NO: 15 - DNA, 987 bp, Arabidopsis thaliana
atggaaaaagccttgagaaacttcaccgaatctacccactcaccagaccctaatcctctcacaaaattcttcactgaacctacagcctcacctgttagccgcaaccgcaaactgtcttcaaaagataccactgtaaccatcgccggagctggcagcagcacgacgaggtaccgcggcgtacgccggaggccgtggggacgatacgcggcggagatacgtgacccaatgtcgaaggagagacgttggctcggaacatttgacacggcggaacaagccgcttgtgcttacgactctgcggctcgtgcctttcgtggagcaaaggctcgtactaattttacttatccgacagctgtcattatgcctgaaccaaggttttctttttccaacaagaaatcttcgccgtctgctcgttgtcctcttccttctctaccgttagattcctctacccaaaacttttacggtgcaccggcagcgcagaggatctataatacacagtctatcttcttacgcgacgcctcgtgttcctctcgtaaaacgactccgtataataactctttcaacggctcatcatcttcttactcagcatcgaaaacggcatgcgtttcttattccgaaaacgaaaacaacgagtcgtttttcccggaagaatcttctgatactggtctattacaagaggtcgttcaagagttcttgaagaaaaatcgcggcgttcctccttctccaccaacaccaccgccggtgactagccatcatgacaactctggttatttctctaatctcactatatactctgaaaatatggttcaagagactaaggagactttgtcgtcgaaactagatcgctacgggaattttcaagctaatgacgacggcgtaagagccgtcgcagacggtggtttatcgttgggatcaaacgagtgggggtatcaagaaatgttgatgtacggaactcagttaggctgtacttgccgaagatcgtggggatag

SEQ ID NO: 16 - DNA, 990 bp, Physcomitrella patens
atgacaaagtcagttcccctgacttcattaatccatggttatgcgattctcaggactgatctcgataccttggagccgttgcaagggatacattggaaatcaagtcgattgatcgaaaacaggcagagcaacggcatggaatctgaatctaggttaggtcgaatgatggacatgacacctttggggtcgggattgcaagggcaacctgttcctggtggagctgcgctcggccttgggccttcgttggagaattcgttgccgcaacccatgtacactcgggggtctgggcaggtaatgacagaagagcagctcgaaacattgcgacgacagatttcggtgtatgcaacaatctgtcaacaacttgttgaaatgcacaaagcgagtgtttcacaacaagcatctcttcctggcattctagcaagtggtcagattgtgtcgatggaccatctcactggaacaccccctcacaaatcgacagcaagacagcggtggacccccagccaacatcagctgcaaattttagaaaagttgtttgagcaaggcagtggcacacccaacaaacagcgcattaaagagattactgccgaactcagtcagcatggtgcaatctcggagacaaatgtgtacaactggtttcagaatcgcaaagcccgagccaaaaggaagcagcaattggttaccccaagggatggtgaatcggaagcagatacagatgtagagtcaccaaaggaaaaacgtacaagacaggaaggtgaacaaaatcaggacgaatcagggggtgttggtgatacaaatggtggaggcaactctgatggagctggaaatggggttcctgagcaaagagctgccaactttgaccagcaggatgccgcttcgtctgcgctgctgcattcacaaacagatactaaacctgatatatcatcatttaacaggagtgctgggttcgatcctcataatgtatctcaaggcatccctcccatgatgagttaa

SEQ ID NO: 17 - DNA, 1190 bp, Arabidopsis thaliana
atggaaaaagccttgagaaacttcaccgaatctacccactcaccagaccctaatcctctcacaaaattcttcactgaacctacagcctcacctgttagccgcaaccgcaaactgtcttcaaaagataccactgtaaccatcgccggagctggcagcagcacgacgaggtaccgcggcgtacgccggaggccgtggggacgatacgcggcggagatacgtgacccaatgtcgaaggagagacgttggctcggaacatttgacacggcggaacaagccgcttgtgcttacgactctgcggctcgtgcctttcgtggagcaaaggctcgtactaattttacttatccgacagctgtcattatgcctgaaccaaggttttctttttccaacaagaaatcttcgccgtctgctcgttgtcctcttccttctctaccgttagattcctctacccaaaacttttacggtgcaccggcagcgcagaggatctataatacacagtctatcttcttacgcgacgcctcgtgttcctctcgtaaaacgactccgtataataactctttcaacggctcatcatcttcttactcagcatcgaaaacggcatgcgtttcttattccgaaaacgaaaacaacgagtcgtttttcccggaagaatcttctgatactggtctattacaagaggtcgttcaagagttcttgaagaaaaatcgcggcgttcctccttctccaccaacaccaccgccggtgactagccatcatgacaactctggttatttctctaatctcactatatactctgaaaatatggttcaagagactaaggagactttgtcgtcgaaactagatcgctacgggaattttcaagctaatgacgacggcgtaagagccgtcgcagacggtggtttatcgttgggatcaaacgagtgggggtatcaagaaatgttgatgtacggaactcagttaggctgtacttgccgaagatcgtggggatagctagatattcatcatgattatgttttgagttttggtactatcgacttagtttaaagttgctacctttcccaatgttggatattaactaaattatgttttaagttgaatttgctaataggatttcataattataatcaagtttataatatattttcgtagctaattaaagtttatatccacgtattctgacacattacgcgctt

SEQ ID NO: 18 - protein, 710 aa, Zea maize
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 19 - protein, 707 aa, Glycine max
MGSMNLLGFSLSPHEEHPSSQDHSQTTPSRFSFNPDGSISSTDVAGGCFDLTSDSTPHLLNLPSYGIYEAFHRNNSINTTQDWKENYNSQNLLLGTSCNKQNMNQNQQQQPKLENFLGGHSFGEHEQTYGGNSASTDYMFPAQPVSAGGGGSGGGSNNNNNSNSIGLSMIKTWLRNQPPNSENINNNNESGGNIRSSVQQTLSLSMSTGSQSSTSLPLLTASVDNGESSSDNKQPNTSAALDSTQTGAIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELSVDNGHRADQVDHSIIMSSHLTQGINNNYAGGGTATHHNWHNAHAFHQPQPCTTMHYPYGQRINWCKQEQQDNSDAPHSLSYSDIHQLQLGNNGTHNFFHTNSGLHPMLSMDSASIDNSSSSNSVVYDGYGGGGGYNVMPMGTTTAVVASDGDQNPRSNHGFGDNEIKALGYESVYGSATDSYHAHARNLYYLTQQQSSSVDTVKASAYDQGSACNTWVPTAIPTHAPRSTTSMALCHGATTPFSLLHE

SEQ ID NO: 20 - protein, 296 aa, Glycine ma
MMEPQQQQQQAQGSQQQQQNEDGGSGKGGFLSRQSSTRWTPTNDQIRILKELYYNNGIRSPSAEQIQRISARLRQYGKIEGKNVFYWFQNHKARERQKKRFTSDHNHNNVPMQRPPTNPSAAWKPDLADPIHTTKYCNISSTAGISSASSSVEMVTVGQMGNYGYGSVPMEKSFRDCSISAGGSSGHVGLINHNLGWVGVDPYNSSTYANFFDKIRPSDQETLEEEAENIGATKIETLPLFPMHGEDIHGYCNLKSNSYNYDGNGWYHTEEGFKNASRASLELSLNSYTRRSPDYA

SEQ ID NO: 21 - protein, 302 aa, Zea maize
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 22 - DNA, 20 bp, Glycine max
gcaaaatatttggctgatgc

SEQ ID NO: 23 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 24 - protein, 1368 aa, Streptococcus pyogenes [sequence omitted]